(19) United States
(12) Patent Application Publication
(10) Pub. No.: US 2026/0252651 A1
(43) Pub. Date: Aug. 27, 2026

(54) SYSTEM

(71) Applicant: SoftBank Group Corp., Tokyo (JP)

(72) Inventor: Tomoharu TSUJI, Tokyo (JP)

(73) Assignee: SoftBank Group Corp., Tokyo (JP)

(21) Appl. No.: 19/537,616

(22) Filed: Feb. 12, 2026

(30) Foreign Application Priority Data
Feb. 21, 2025 (JP) 2025-027021

(51) Int. Cl. G06F 16/9535; G06F 16/2457; G16H 20/60

(52) U.S. Cl. CPC G06F 16/9535; G06F 16/24578; G16H 20/60

(57) ABSTRACT

The system according to the embodiment comprises a collection unit, a proposal unit, and a reception unit. The collection unit collects user information. The proposal unit proposes an appropriate meal plan based on the information collected by the collection unit. The reception unit receives user feedback on the meal plan proposed by the proposal unit.

### CROSS-REFERENCE TO RELATED APPLICATION

The present application claims priority to and incorporates by reference the entire contents of Japanese Patent Application No. 2025-027021 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there has been a problem that it is difficult to provide meal plans tailored to each user's individual nutritional needs and taste preferences.

### SUMMARY OF THE INVENTION

The system according to the embodiment comprises a collection unit, a proposal unit, and a reception unit. The collection unit collects user information. The proposal unit proposes an appropriate meal plan based on the information collected by the collection unit. The reception unit receives user feedback on the meal plan proposed by the proposal unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

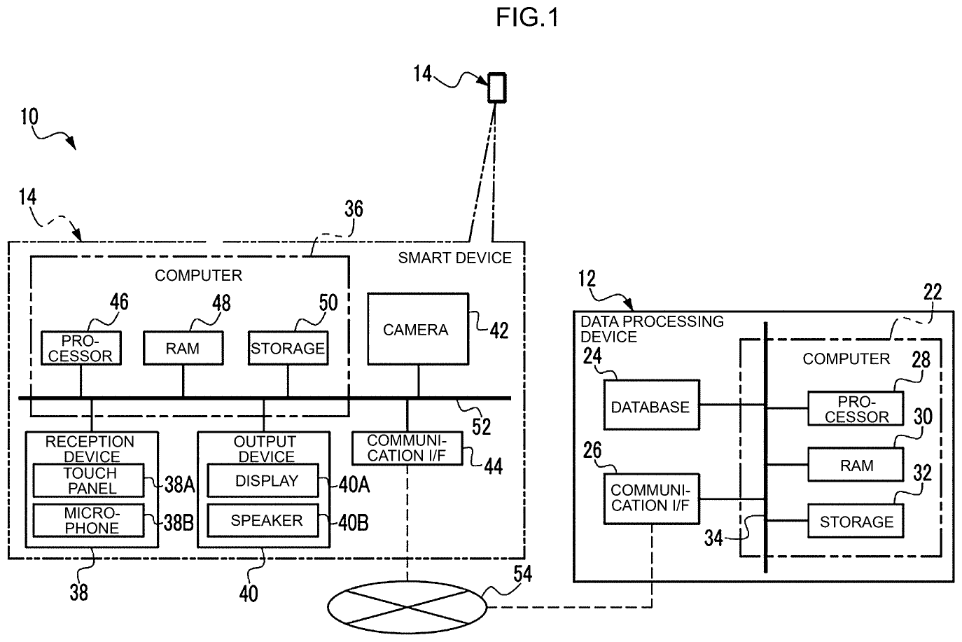

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

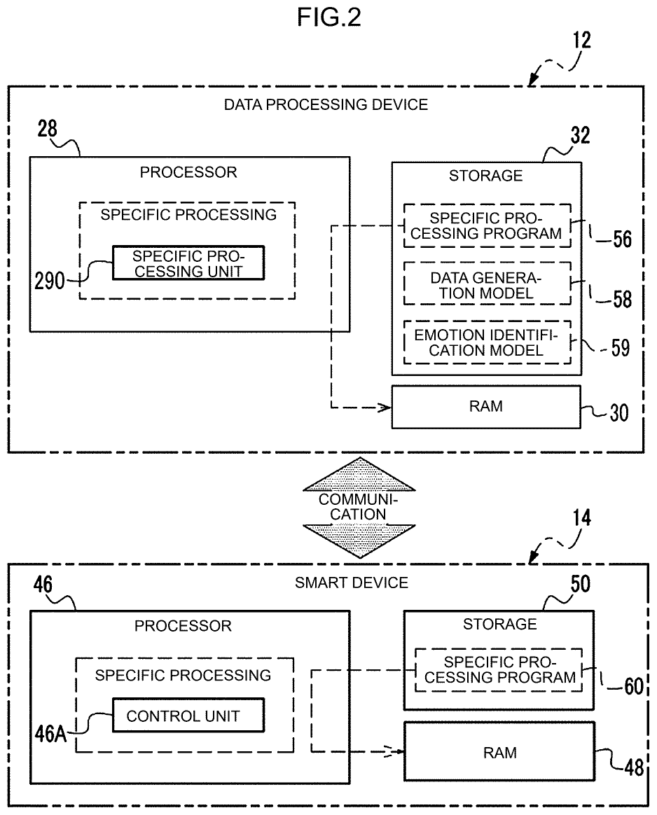

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The NutriAI system according to the embodiment of the present invention is a system that provides personalized meal plans tailored to each user's nutritional needs, taste preferences, and health status. The NutriAI system learns the user's lifestyle and proposes optimal meals based on individual profiles. As a result, users can enjoy their own healthy and well-balanced meals and receive support for leading a healthier life. For example, the NutriAI system allows users to input information such as their lifestyle, nutritional needs, taste preferences, and health status. Next, AI learns this information and proposes optimal meal plans based on individual profiles. For instance, if the user is on a diet, the system proposes low-calorie and nutritionally balanced meals. If the user has specific allergies, the system proposes meals that do not contain those allergens. Furthermore, the system selects meal recipes and ingredients according to the user's taste preferences. This enables users to enjoy their own healthy and well-balanced meals and receive support for a healthier lifestyle. For example, for busy businesspersons, the system proposes recipes that can be prepared in a short time to help maintain a healthy diet. For athletes, the system proposes meals containing nutrients necessary for training to support performance improvement. In this way, the NutriAI system provides personalized meal plans tailored to diverse user needs and supports healthy living. Thus, the NutriAI system can provide personalized meal plans tailored to users' nutritional needs, taste preferences, and health status. Specifically, the NutriAI system stores information collected from users (e.g., age, gender, height, weight, medical history, allergies, meal history, exercise habits, lifestyle rhythm, preferences, geographic location information, social media activity, etc.) as multidimensional vectors in a database. The collection unit of the system performs preprocessing such as normalization, categorization, and quantification of this information before inputting it into a neural network-based AI model (e.g., Transformer-based large language models or multimodal models). The AI model receives input vectors (e.g., age=35, gender=female, allergy=wheat, diet goal=weight—5 kg, preference=Japanese cuisine, exercise habit=running three times a week, etc.) and generates structured data for meal plans as output (e.g., breakfast=oatmeal+yogurt, lunch=chicken breast salad, dinner=grilled salmon+vegetable soup, calorie and nutrient distribution for each meal, cooking time, recommended recipe URLs, etc.). To consider the user's health status and allergy information when generating meal plans, the AI model incorporates penalty terms such as “allergen avoidance,” “nutritional balance,” and “calorie constraints” into the loss function, thereby achieving optimization in high-dimensional space, which is different from conventional simple recommendations. Furthermore, the system collects user feedback (e.g., satisfaction scores, meal implementation status, taste evaluation, requests for improvement, etc.) via the reception unit and has an online learning function that sequentially updates the AI model weights using this feedback as retraining data. This personalizes the model for each user and continuously improves proposal accuracy. As a technical effect, the system enables large-scale data integration analysis, rapid optimization, and proposal generation under diverse constraints, which are impossible by manual work of human nutritionists, and can provide optimized meal plans for each user in real time, thereby delivering remarkable effects such as improved health management accuracy, diversification of meal proposals, increased user satisfaction, and reduced system operation costs. Specific application fields include personal health management apps, corporate welfare systems, nutritional guidance support in medical institutions, meal management for sports teams, and meal optimization in nursing care facilities.

The NutriAI system according to the embodiment comprises a collection unit, a proposal unit, and a reception unit. The collection unit collects user information. User information may include, for example, age, gender, health status, and dietary preferences, but is not limited to these examples. The collection unit, for example, stores information input by the user in a database and converts it into a format that is easy for AI to analyze. The proposal unit proposes an optimal meal plan based on the information collected by the collection unit. The proposal unit, for example, uses AI to generate meal plans based on the user's nutritional needs, taste preferences, and health status. The proposal unit uses generative AI (e.g., text generation AI or multimodal generative AI) to propose optimal meal plans to the user. For example, if the user is on a diet, the proposal unit proposes low-calorie and nutritionally balanced meals. If the user has specific allergies, the proposal unit proposes meals that do not contain those allergens. Furthermore, the proposal unit selects meal recipes and ingredients according to the user's taste preferences. The reception unit receives user feedback on the meal plan proposed by the proposal unit. The reception unit, for example, allows the user to input satisfaction or points for improvement regarding the proposed meal plan. The reception unit uses AI to analyze user feedback and provides feedback to the proposal unit. As a result, the proposal unit can improve the meal plan based on user feedback. Thus, the NutriAI system according to the embodiment can provide personalized meal plans by collecting user information, proposing optimal meal plans, and receiving feedback. Specifically, the NutriAI system stores information obtained by the collection unit from the user (e.g., age, gender, height, weight, medical history, allergies, meal history, exercise habits, lifestyle rhythm, preferences, geographic location information, social media activity, etc.) as multidimensional vectors (e.g., each element quantified/categorized as a 128-dimensional vector) in a database. The collection unit performs preprocessing such as normalization (e.g., scaling age to 0-1), categorization (e.g., one-hot encoding for gender), and quantification (e.g., converting meal history to frequency vectors) before inputting the data into a neural network-based AI model (e.g., Transformer-based large language models or multimodal models). The AI model receives input vectors (e.g., age=35, gender=female, allergy=wheat, diet goal=weight—5 kg, preference=Japanese cuisine, exercise habit=running three times a week, etc.) and generates structured data for meal plans as output (e.g., breakfast=oatmeal+yogurt, lunch=chicken breast salad, dinner=grilled salmon+vegetable soup, calorie and nutrient distribution for each meal, cooking time, recommended recipe URLs, etc.). To consider the user's health status and allergy information when generating meal plans, the AI model incorporates penalty terms such as “allergen avoidance,” “nutritional balance,” and “calorie constraints” into the loss function, thereby achieving optimization in high-dimensional space, which is different from conventional simple recommendations. For example, input examples include “age=28, gender=male, allergy=egg, preference=Italian, exercise habit=going to the gym twice a week” or “age=60, gender=female, medical history=hypertension, preference=Japanese cuisine, exercise habit=walking.” Output examples include “breakfast=tomato and basil salad, lunch=grilled chicken and vegetables, dinner=braised fish+brown rice” or “breakfast=natto rice, lunch=grilled mackerel, dinner=miso soup with plenty of vegetables.” The reception unit receives user feedback (e.g., satisfaction scores, meal implementation status, taste evaluation, requests for improvement, etc.) via a feedback screen or voice input and has an online learning function that sequentially updates the AI model weights using this feedback as retraining data. The AI model uses cross-entropy loss and custom penalty terms as the loss function and updates weights using gradient descent or Adam optimization. This personalizes the model for each user and continuously improves proposal accuracy. As a technical effect, the system enables large-scale data integration analysis, rapid optimization, and proposal generation under diverse constraints, which are impossible by manual work of human nutritionists, and can provide optimized meal plans for each user in real time, thereby delivering remarkable effects such as improved health management accuracy, diversification of meal proposals, increased user satisfaction, and reduced system operation costs. Specific application fields include personal health management apps, corporate welfare systems, nutritional guidance support in medical institutions, meal management for sports teams, and meal optimization in nursing care facilities.

The proposal unit can propose an appropriate meal plan based on the user's age, gender, weight, height, allergy information, dietary preferences, and exercise habits. The proposal unit, for example, collects the user's age, gender, weight, height, allergy information, dietary preferences, and exercise habits, and proposes an optimal meal plan based on this information. For example, if the user is on a diet, the proposal unit proposes low-calorie and nutritionally balanced meals. If the user has specific allergies, the proposal unit proposes meals that do not contain those allergens. Furthermore, the proposal unit can propose meals containing nutrients necessary for training based on the user's exercise habits. Thus, the proposal unit can propose more appropriate meal plans based on detailed user information. Some or all of the above-described processing in the proposal unit may be performed using AI or without using AI. For example, the proposal unit can use an AI model that receives user information as input and outputs an optimal meal plan to propose meal plans. Specifically, the proposal unit normalizes, categorizes, and quantifies information such as age, gender, weight, height, allergy information, dietary preferences, and exercise habits collected from the user as a 128-dimensional multidimensional vector and stores it in a database. The proposal unit inputs these vector data into Transformer-based large language models or multimodal models. Input examples include “age=30, gender=male, weight=70 kg, height=175 cm, allergy=dairy, preference=Japanese cuisine, exercise habit=going to the gym twice a week” or “age=45, gender=female, weight=60 kg, height=160 cm, allergy=none, preference=Italian, exercise habit=doing yoga once a week.”The AI model receives these input vectors and generates structured data for meal plans as output (e.g., breakfast=natto rice+miso soup, lunch=chicken breast salad, dinner=grilled salmon+vegetable soup, calorie and nutrient distribution for each meal, cooking time, recommended recipe URLs, etc.). Output examples include “breakfast=oatmeal+yogurt, lunch=grilled chicken and vegetables, dinner=braised fish+brown rice” or “breakfast=tomato and basil salad, lunch=grilled mackerel, dinner=miso soup with plenty of vegetables.” The proposal unit incorporates penalty terms such as “allergen avoidance,” “nutritional balance,” and “calorie constraints” into the loss function of the AI model, thereby achieving optimization in high-dimensional space, which is different from conventional rule-based or experience-based proposals. The AI model uses cross-entropy loss and custom penalty terms and updates weights using gradient descent or Adam optimization. Furthermore, the proposal unit can implement an algorithm that automatically adjusts the distribution of protein and carbohydrates according to training intensity and frequency based on the user's exercise habit information. As a result, the proposal unit can generate optimized meal plans for each user in real time, delivering remarkable technical effects such as improved health management accuracy, diversification of meal proposals, increased user satisfaction, and reduced system operation costs. Specific application fields include personal health management apps, corporate welfare systems, nutritional guidance support in medical institutions, meal management for sports teams, and meal optimization in nursing care facilities.

The reception unit receives user feedback, and the proposal unit can improve the meal plan based on that feedback. The reception unit, for example, allows the user to input satisfaction or points for improvement regarding the proposed meal plan. The reception unit uses AI to analyze user feedback and provides feedback to the proposal unit. The proposal unit improves the meal plan based on user feedback. For example, if the user is dissatisfied with the proposed meal plan, the proposal unit analyzes the reason and reflects it in the next proposal. Furthermore, if the user dislikes certain ingredients, the proposal unit can propose meal plans that do not include those ingredients. Thus, the proposal unit can provide more personalized proposals by improving meal plans based on user feedback. Some or all of the above-described processing in the reception unit may be performed using AI or without using AI. For example, the reception unit can use an AI model that receives user feedback as input and analyzes the feedback to provide it to the proposal unit. Specifically, the reception unit normalizes and categorizes feedback collected from users (e.g., satisfaction scores, meal implementation status, taste evaluation, requests for improvement, free-text comments, etc.) as text or numerical data and converts it into a format that is easy for the AI model to analyze. Input examples include “satisfaction=3/5, meal implementation=not possible, reason=taste is bland” or “satisfaction=5/5, meal implementation=possible, comment=very delicious.” The reception unit inputs these feedback data into Transformer-based large language models or multimodal models and generates structured data as output, such as “elements to be improved (e.g., seasoning strength, ingredient type, cooking time)” or “parameter adjustment values for the next proposal (e.g., salt+10%, cooking time—5 minutes).” Output examples include “make the seasoning stronger next time,” “avoid chicken next time,” or “focus on Japanese cuisine next time.” The reception unit incorporates penalty terms such as “user satisfaction improvement” and “feedback reflection rate” into the loss function of the AI model, thereby achieving optimization in high-dimensional space, which is different from conventional simple questionnaire aggregation or experience-based improvements. The AI model uses cross-entropy loss and custom penalty terms and updates weights using gradient descent or Adam optimization. Furthermore, the reception unit has an online learning function for each user and personalizes the model sequentially, thereby continuously improving proposal accuracy. As a result, the reception unit can realize optimized feedback analysis and proposal improvement for each user in real time, delivering remarkable technical effects such as increased user satisfaction, reduced system operation costs, and improved health management accuracy. Specific application fields include personal health management apps, corporate welfare systems, nutritional guidance support in medical institutions, meal management for sports teams, and meal optimization in nursing care facilities.

The proposal unit can select recipes and ingredients tailored to the user's lifestyle. The proposal unit, for example, selects recipes and ingredients tailored to the user's lifestyle. For example, for busy businesspersons, the proposal unit proposes recipes that can be prepared in a short time. For athletes, the proposal unit can propose meals containing nutrients necessary for training. Furthermore, the proposal unit can select meal recipes and ingredients according to the user's taste preferences. Thus, the proposal unit can provide more appropriate meal plans by selecting recipes and ingredients tailored to the user's lifestyle. Some or all of the above-described processing in the proposal unit may be performed using AI or without using AI. For example, the proposal unit can use an AI model that receives user lifestyle information as input and outputs optimal recipes and ingredients to select recipes and ingredients. Specifically, the proposal unit normalizes, categorizes, and quantifies lifestyle information collected from users (e.g., occupation, working hours, family structure, cooking skills, number of meals, frequency of eating out, exercise habits, hobbies, taste preferences, etc.) as multidimensional vectors and stores them in a database. The proposal unit inputs these vector data into Transformer-based large language models or multimodal models. Input examples include “occupation=company employee, working hours=9-18, family structure=single, cooking skill=beginner, frequency of eating out=three times a week, preference=Japanese cuisine” or “occupation=athlete, exercise habit=training every day, cooking skill=advanced, preference=high protein.” The AI model receives these input vectors and generates structured data for recipes and ingredients as output (e.g., recipe name, cooking time, required ingredient list, nutrient distribution, cooking procedure, recommended cooking utensils, etc.). Output examples include “recipe=grilled chicken breast, cooking time=15 minutes, ingredients=chicken breast, broccoli, olive oil” or “recipe=natto rice, cooking time=5 minutes, ingredients=natto, rice, green onion.” The proposal unit incorporates penalty terms such as “cooking time constraints,” “nutritional balance,” and “taste preference fit” into the loss function of the AI model, thereby achieving optimization in high-dimensional space, which is different from conventional simple recipe searches or experience-based proposals. The AI model uses cross-entropy loss and custom penalty terms and updates weights using gradient descent or Adam optimization. Furthermore, the proposal unit can personalize the model sequentially using online learning functions according to changes in the user's lifestyle (e.g., job change, family structure change, change in exercise habits, etc.). As a result, the proposal unit can generate optimized recipe and ingredient proposals for each user in real time, delivering remarkable technical effects such as improved health management accuracy, diversification of meal proposals, increased user satisfaction, and reduced system operation costs. Specific application fields include personal health management apps, corporate welfare systems, nutritional guidance support in medical institutions, meal management for sports teams, and meal optimization in nursing care facilities.

The proposal unit can propose low-calorie and nutritionally balanced meals when the user is on a diet. The proposal unit, for example, proposes low-calorie and nutritionally balanced meals when the user is on a diet. For example, the proposal unit proposes meal plans considering calorie restrictions. The proposal unit can also propose meal plans considering nutrient distribution. Furthermore, the proposal unit can propose low-calorie and nutritionally balanced meals according to the user's taste preferences. Thus, the proposal unit can provide appropriate meal plans for users on a diet. Some or all of the above-described processing in the proposal unit may be performed using AI or without using AI. For example, the proposal unit can use an AI model that receives user diet information as input and outputs low-calorie and nutritionally balanced meal plans to propose meal plans. Specifically, the proposal unit normalizes, categorizes, and quantifies diet information collected from users (e.g., target weight, target period, current weight and height, basal metabolic rate, activity level, meal history, taste preferences, etc.) as multidimensional vectors and stores them in a database. The proposal unit inputs these vector data into Transformer-based large language models or multimodal models. Input examples include “target weight=60 kg, target period=3 months, current weight=65 kg, height=165 cm, activity level=medium, preference=Japanese cuisine” or “target weight=70 kg, target period=2 months, current weight=75 kg, height=180 cm, activity level=high, preference=Italian.” The AI model receives these input vectors and generates structured data for low-calorie and nutritionally balanced meal plans as output (e.g., breakfast=oatmeal+yogurt, lunch=chicken breast salad, dinner=grilled salmon+vegetable soup, calorie and nutrient distribution for each meal, cooking time, recommended recipe URLs, etc.). Output examples include “breakfast=natto rice, lunch=grilled chicken and vegetables, dinner=braised fish+brown rice” or “breakfast=tomato and basil salad, lunch=grilled mackerel, dinner=miso soup with plenty of vegetables.” The proposal unit incorporates penalty terms such as “calorie constraints,” “nutritional balance,” and “taste preference fit” into the loss function of the AI model, thereby achieving optimization in high-dimensional space, which is different from conventional simple calorie calculations or experience-based proposals. The AI model uses cross-entropy loss and custom penalty terms and updates weights using gradient descent or Adam optimization. Furthermore, the proposal unit sequentially learns user feedback and weight change data to personalize the model, thereby continuously improving proposal accuracy. As a result, the proposal unit can generate optimized meal plans for each user on a diet in real time, delivering remarkable technical effects such as improved health management accuracy, diversification of meal proposals, increased user satisfaction, and reduced system operation costs. Specific application fields include personal diet support apps, weight loss guidance in medical institutions, nutrition management in sports gyms, and corporate health management support.

The proposal unit can propose meals containing nutrients necessary for training to athletes. The proposal unit, for example, proposes meals containing nutrients necessary for training to athletes. For example, the proposal unit proposes meal plans considering nutrients such as protein, carbohydrates, and vitamins. The proposal unit can also propose meal plans containing optimal nutrients according to the type and intensity of training. Furthermore, the proposal unit can propose meals containing nutrients necessary for training according to the athlete's taste preferences. Thus, the proposal unit can provide appropriate meal plans containing nutrients for athletes. Some or all of the above-described processing in the proposal unit may be performed using AI or without using AI. For example, the proposal unit can use an AI model that receives athlete training information as input and outputs meal plans containing nutrients necessary for training to propose meal plans. Specifically, the proposal unit normalizes, categorizes, and quantifies training information collected from athletes (e.g., sport type, training frequency, intensity, goals, weight and height, taste preferences, allergy information, etc.) as multidimensional vectors and stores them in a database. The proposal unit inputs these vector data into Transformer-based large language models or multimodal models. Input examples include “sport type=soccer, training frequency=five times a week, intensity=high, weight=70 kg, preference=Japanese cuisine” or “sport type=swimming, training frequency=daily, intensity=medium, weight=65 kg, preference=Italian.” The AI model receives these input vectors and generates structured data for meal plans containing nutrients necessary for training as output (e.g., breakfast=high-protein omelet+whole grain bread, lunch=chicken breast salad+brown rice, dinner=grilled salmon+vegetable soup, calorie, protein, carbohydrate, fat, vitamin distribution for each meal, cooking time, recommended recipe URLs, etc.). Output examples include “breakfast=protein pancakes, lunch=grilled chicken and vegetables, dinner=braised fish+brown rice” or “breakfast=natto rice, lunch=grilled mackerel, dinner=miso soup with plenty of vegetables.” The proposal unit incorporates penalty terms such as “protein target amount,” “energy supply,” “allergen avoidance,” and “taste preference fit” into the loss function of the AI model, thereby achieving optimization in high-dimensional space, which is different from conventional simple nutrition calculations or experience-based proposals. The AI model uses cross-entropy loss and custom penalty terms and updates weights using gradient descent or Adam optimization. Furthermore, the proposal unit sequentially learns changes in training content and body composition data to personalize the model, thereby continuously improving proposal accuracy. As a result, the proposal unit can generate optimized meal plans for each athlete in real time, delivering remarkable technical effects such as improved performance, improved health management accuracy, diversification of meal proposals, increased user satisfaction, and reduced system operation costs. Specific application fields include meal management for sports teams, nutrition guidance for athletes, meal optimization in training gyms, and rehabilitation support in medical institutions.

The collection unit can estimate the user's emotions and adjust the timing of information collection based on the estimated emotions. The collection unit, for example, estimates the user's emotions and adjusts the timing of information collection based on the estimated emotions. For example, if the user is feeling stressed, the collection unit collects information during relaxed periods. If the user is relaxed, the collection unit can take more time to collect detailed information. Furthermore, if the user is busy, the collection unit can collect necessary information in a short time. Thus, the collection unit can adjust the timing of information collection according to the user's emotions, enabling more appropriate information collection. Emotion estimation is realized using, for example, an emotion engine or generative AI with emotion estimation functions. Generative AI may be text generation AI (e.g., LLM) or multimodal generative AI, but is not limited to these examples. Some or all of the above-described processing in the collection unit may be performed using AI or without using AI. For example, the collection unit can use an AI model that receives user emotion data as input and adjusts the timing of information collection. Specifically, the collection unit receives various multimodal data as input for estimating user emotions, such as audio data (e.g., conversation waveform data, sampling rate 16 kHz, 1-minute audio clips), text data (e.g., chat history, SNS posts, up to 512 tokens per post), and image data (e.g., facial expression images, resolution 224×224 pixels, RGB 3 channels). The collection unit performs preprocessing such as normalization (e.g., Mel spectrogram conversion for audio, tokenization for text, resizing and standardization for images) and feature extraction (e.g., audio emotion features, BERT embeddings, facial expression feature extraction using CNN) before inputting the data into a Transformer-based multimodal emotion estimation model. The AI model integrates the input multidimensional tensors (e.g., 128-dimensional audio feature vector, 768-dimensional text embedding, 512-dimensional image feature vector) and generates output such as emotion labels (e.g., stress, relaxation, fatigue, anger, joy, etc.), emotion intensity scores (e.g., continuous values from 0.0 to 1.0), and estimation confidence (e.g., probability distribution). Specific output examples include “emotion=stress, intensity=0.85, confidence=0.92” or “emotion=relaxation, intensity=0.40, confidence=0.88.” Based on these output results, the collection unit applies an information collection timing determination algorithm (e.g., if stress intensity is 0.7 or higher, collect during nighttime relaxation time; if relaxation intensity is 0.5 or higher, present detailed questionnaires; if fatigue intensity is high, reduce collection frequency) to automate scheduling of the information collection process. Furthermore, the collection unit can analyze time-series emotion change patterns using time-series models such as LSTM by linking with past emotion transition data and behavior logs, and predict optimal future collection timing. For AI model training, cross-entropy loss for maximizing emotion estimation accuracy and custom penalty terms for reducing user burden and improving collection efficiency are incorporated, thereby achieving optimization in high-dimensional space, which is different from conventional simple scheduled collection or experience-based timing adjustment. As a technical effect, the collection unit can minimize the user's psychological burden while collecting necessary information with high accuracy and efficiency, thereby delivering remarkable effects such as improved data quality, reduced user dropout rate, and improved overall system operation efficiency. Specific application fields include personal health management apps, mental health care support systems, employee health monitoring in companies, patient status observation in medical institutions, and user status monitoring in nursing care facilities.

The collection unit can analyze the user's past meal history and select an optimal information collection method. The collection unit, for example, analyzes the user's past meal history and selects an optimal information collection method. For example, the collection unit prioritizes collecting related information based on dishes the user has enjoyed in the past. The collection unit can also collect information considering allergy information based on ingredients the user has avoided in the past. Furthermore, if a specific nutrient is found to be lacking from the user's meal history, the collection unit can collect information on ingredients containing that nutrient. Thus, the collection unit can perform more appropriate information collection by analyzing the user's past meal history. Some or all of the above-described processing in the collection unit may be performed using AI or without using AI. For example, the collection unit can use an AI model that receives the user's past meal history data as input and selects an optimal information collection method. Specifically, the collection unit stores the user's past meal history data (e.g., date, meal content, calorie intake, nutrient distribution, ingredient list, intake amount, meal satisfaction score, etc.) as a time-series database and inputs each meal history as a multidimensional vector (e.g., 64 dimensions per meal, 7×64 dimensions tensor for one week) into the AI model. The collection unit performs preprocessing such as normalization (e.g., scaling calories per meal to 0-1), categorization (e.g., one-hot encoding for cuisine genre), and quantification (e.g., converting nutrient intake to real-valued vectors). The AI model may be a Transformer-based time-series analysis model or an LSTM-type recurrent neural network. Input examples include “2024/6/1: breakfast=natto rice, lunch=chicken breast salad, dinner=grilled salmon+vegetable soup, calories, protein, fat, carbohydrate, vitamin amount for each meal” or “2024/6/2: breakfast=oatmeal+yogurt, lunch=grilled chicken and vegetables, dinner=braised fish+brown rice.” The AI model analyzes these history tensors and generates structured data as output, such as “information categories to be prioritized for collection (e.g., Japanese recipes, low-carb ingredients, high-protein ingredients, allergen-free recipes, etc.),” “recommended collection methods (e.g., questionnaire format, image upload, voice input, etc.),” and “collection frequency (e.g., once a week, after every meal).” Output examples include “priority category=high-protein Japanese cuisine, recommended method=image+text, frequency=after every meal” or “priority category=low-carb Italian, recommended method=questionnaire, frequency=twice a week.” The collection unit incorporates penalty terms such as “information collection efficiency,” “user burden reduction,” “allergen avoidance,” and “nutrient sufficiency” into the loss function of the AI model, thereby achieving optimization in high-dimensional space, which is different from conventional simple history reference or experience-based collection design. Furthermore, the collection unit sequentially learns user feedback and new meal history and can personalize the model using online learning functions. As a technical effect, the collection unit can generate optimized information collection designs for each user in real time, thereby delivering remarkable effects such as improved data quality, increased user satisfaction, and reduced system operation costs. Specific application fields include personal health management apps, nutritional guidance support in medical institutions, meal management for sports teams, and meal history monitoring in nursing care facilities.

The collection unit can perform filtering based on the user's current health status and lifestyle at the time of information collection. The collection unit, for example, performs filtering based on the user's current health status and lifestyle at the time of information collection. For example, the collection unit collects information on ingredients containing specific nutrients if the user needs them based on health check results. The collection unit can also collect information on appropriate meal times based on the user's lifestyle (e.g., night type, morning type). Furthermore, the collection unit can collect information on meals suitable after exercise based on the user's exercise habits. Thus, the collection unit can perform more appropriate information collection by filtering information based on the user's health status and lifestyle. Some or all of the above-described processing in the collection unit may be performed using AI or without using AI. For example, the collection unit can use an AI model that receives user health status and lifestyle data as input and performs information collection by filtering information. Specifically, the collection unit stores user health check data (e.g., blood test values, BMI, blood pressure, medical history, doctor comments, etc.) and lifestyle data (e.g., wake-up and bedtime, number of meals, frequency of eating out, exercise frequency and intensity, smoking and drinking habits, etc.) as multidimensional vectors (e.g., 128 dimensions) in a database and performs preprocessing such as normalization (e.g., scaling blood pressure to 0-1), categorization (e.g., one-hot encoding for lifestyle rhythm as morning type or night type), and quantification (e.g., converting exercise frequency to real values per week). The collection unit inputs these vector data into Transformer-based large language models or multimodal models. Input examples include “blood pressure=130/85, BMI=24, medical history=hypertension, lifestyle rhythm=night type, exercise frequency=twice a week” or “blood test=anemia tendency, lifestyle rhythm=morning type, exercise frequency=daily.” The AI model analyzes these input vectors and generates structured data as output, such as “information categories to be collected (e.g., iron-rich ingredients, low-salt recipes, breakfast recipes, post-exercise meals, etc.)” and “recommended collection timing (e.g., before breakfast, after exercise, at night).” Output examples include “category=iron-fortified recipes, timing=before breakfast” or “category=low-salt Japanese cuisine, timing=at dinner.” The collection unit incorporates penalty terms such as “health status fit,” “lifestyle fit,” and “information collection efficiency” into the loss function of the AI model, thereby achieving optimization in high-dimensional space, which is different from conventional simple health check reference or experience-based filtering. Furthermore, the collection unit sequentially learns changes in the user's health status and lifestyle and can personalize the model using online learning functions. As a technical effect, the collection unit can generate optimized information collection designs for each user in real time, thereby delivering remarkable effects such as improved health management accuracy, improved data quality, increased user satisfaction, and reduced system operation costs. Specific application fields include personal health management apps, nutritional guidance support in medical institutions, meal management for sports teams, and health status monitoring in nursing care facilities.

The collection unit can estimate the user's emotions and determine the priority of information to be collected based on the estimated emotions. The collection unit, for example, estimates the user's emotions and determines the priority of information to be collected based on the estimated emotions. For example, if the user is feeling stressed, the collection unit prioritizes collecting information on ingredients with relaxing effects. If the user is relaxed, the collection unit prioritizes collecting information on new recipes and ingredients. Furthermore, if the user is tired, the collection unit prioritizes collecting information on ingredients suitable for energy replenishment. Thus, the collection unit can perform more appropriate information collection by determining the priority of information according to the user's emotions. Emotion estimation is realized using, for example, an emotion engine or generative AI with emotion estimation functions. Generative AI may be text generation AI (e.g., LLM) or multimodal generative AI, but is not limited to these examples. Some or all of the above-described processing in the collection unit may be performed using AI or without using AI. For example, the collection unit can use an AI model that receives user emotion data as input and determines the priority of information to be collected. Specifically, the collection unit receives multimodal data for emotion estimation, such as audio data (e.g., conversation waveform data, sampling rate 16 kHz), text data (e.g., chat history, SNS posts, up to 512 tokens per post), and image data (e.g., facial expression images, resolution 224×224 pixels) as input. The collection unit performs preprocessing such as normalization and feature extraction (e.g., audio emotion features, BERT embeddings, facial expression feature extraction using CNN) before inputting the data into a Transformer-based multimodal emotion estimation model. The AI model integrates the input multidimensional tensors (e.g., 128-dimensional audio feature vector, 768-dimensional text embedding, 512-dimensional image feature vector) and generates output such as emotion labels (e.g., stress, relaxation, fatigue, etc.), emotion intensity scores (e.g., 0.0-1.0), and estimation confidence (e.g., probability distribution). Output examples include “emotion=stress, intensity=0.80” or “emotion=relaxation, intensity=0.60.” Based on these output results, the collection unit applies an algorithm to calculate priority scores for each information category (e.g., if stress intensity is high, set priority for relaxing ingredient information to 1.0; if fatigue intensity is high, set priority for energy replenishment ingredient information to 0.9, etc.) and automatically determines the priority of the information collection process. Furthermore, the collection unit can perform time-series priority adjustment using time-series models such as LSTM by linking with past emotion transitions and feedback data. For AI model training, cross-entropy loss for maximizing emotion estimation and priority determination accuracy and custom penalty terms for improving user satisfaction are incorporated, thereby achieving optimization in high-dimensional space, which is different from conventional rule-based or experience-based priority determination. As a technical effect, the collection unit can optimize information collection in real time according to the user's psychological state, thereby delivering remarkable effects such as improved data quality, increased user satisfaction, and improved system operation efficiency. Specific application fields include personal health management apps, mental health care support systems, patient status observation in medical institutions, and user status monitoring in nursing care facilities.

The collection unit can preferentially collect highly relevant information based on the user's geographic location at the time of information collection. The collection unit, for example, preferentially collects highly relevant information based on the user's geographic location at the time of information collection. For example, the collection unit collects information on local specialties and seasonal ingredients in the user's residential area. The collection unit can also collect information on local food culture and recommended restaurants if the user is traveling. Furthermore, if the user plans to move to a specific region, the collection unit can collect information on ingredients and recipes from that region. Thus, the collection unit can collect more relevant information by considering the user's geographic location. Some or all of the above-described processing in the collection unit may be performed using AI or without using AI. For example, the collection unit can use an AI model that receives user geographic location information as input and preferentially collects highly relevant information. Specifically, the collection unit stores user geographic location information (e.g., GPS coordinates, prefecture/city codes, movement history, length of stay, etc.) as multidimensional vectors (e.g., 16 dimensions including latitude, longitude, region category, movement frequency, etc.) in a database and performs preprocessing such as normalization (e.g., scaling latitude and longitude to 0-1), categorization (e.g., one-hot encoding for region category), and quantification (e.g., converting movement frequency to real values per week). The collection unit inputs these vector data into Transformer-based large language models or multimodal models. Input examples include “current location=Chiyoda-ku, Tokyo, movement history=Osaka, Kyoto, length of stay=3 days” or “current location=Sapporo, Hokkaido, movement history=none, length of stay=1 year.” The AI model analyzes these input vectors and generates structured data as output, such as “information categories to be prioritized for collection (e.g., seasonal vegetables from Hokkaido, local cuisine recipes from Sapporo, recommended restaurants in Chiyoda-ku, etc.)” and “recommended collection methods (e.g., local surveys, image upload, region-limited recipe search, etc.).” Output examples include “priority category=Sapporo local cuisine, recommended method=recipe image collection” or “priority category=Osaka specialties, recommended method=local survey.” The collection unit incorporates penalty terms such as “regional relevance,” “information collection efficiency,” and “user satisfaction” into the loss function of the AI model, thereby achieving optimization in high-dimensional space, which is different from conventional simple region filters or experience-based information collection. Furthermore, the collection unit sequentially learns changes in the user's movement history and regional preferences and can personalize the model using online learning functions. As a technical effect, the collection unit can optimize information collection in real time according to the user's geographic situation, thereby delivering remarkable effects such as improved information relevance, increased user satisfaction, and improved system operation efficiency. Specific application fields include personal health management apps, meal proposal systems for travelers, regional specialty promotion, support for using local ingredients in medical institutions, and introduction of regional food culture in nursing care facilities.

The collection unit can analyze the user's social media activity at the time of information collection and collect relevant information. The collection unit, for example, analyzes the user's social media activity at the time of information collection and collects relevant information. For example, the collection unit collects information on related recipes and ingredients based on meal photos shared by the user on social media. The collection unit can also analyze posts from cooking accounts followed by the user and collect information that may be of interest. Furthermore, the collection unit can analyze trends in cooking communities the user participates in and collect related information. Thus, the collection unit can collect more relevant information by analyzing the user's social media activity. Some or all of the above-described processing in the collection unit may be performed using AI or without using AI. For example, the collection unit can use an AI model that receives user social media data as input and collects relevant information. Specifically, the collection unit stores user social media activity data (e.g., post text, images, videos, like history, followed accounts, participating communities, post date and time, etc.) as multidimensional vectors (e.g., 768-dimensional text embeddings, 512-dimensional image feature vectors, 32-dimensional account category vectors, etc.) in a database and performs preprocessing such as normalization (e.g., scaling post frequency to 0-1), categorization (e.g., one-hot encoding for account type), and quantification (e.g., converting number of likes to real values). The collection unit inputs these vector data into Transformer-based multimodal models. Input examples include “post text=‘Today's lunch is salmon bowl,’ image=salmon bowl photo, followed account=Japanese recipe, post date=2024/6/1” or “post text=‘Trying a new pasta recipe,’ image=pasta photo, followed account=Italian cuisine.” The AI model analyzes these input vectors and generates structured data as output, such as “information categories to be prioritized for collection (e.g., Japanese recipes, salmon dishes, Italian pasta, etc.)” and “recommended collection methods (e.g., image analysis, text mining, community trend analysis, etc.).” Output examples include “priority category=salmon recipes, recommended method=image analysis” or “priority category=Italian pasta, recommended method=text mining.” The collection unit incorporates penalty terms such as “information relevance,” “user interest fit,” and “information collection efficiency” into the loss function of the AI model, thereby achieving optimization in high-dimensional space, which is different from conventional simple keyword searches or experience-based information collection. Furthermore, the collection unit sequentially learns changes in the user's social media activity and new trends and can personalize the model using online learning functions. As a technical effect, the collection unit can optimize information collection in real time according to the user's interests and preferences, thereby delivering remarkable effects such as improved information relevance, increased user satisfaction, and improved system operation efficiency. Specific application fields include personal health management apps, recipe proposal services, meal trend analysis, corporate marketing support, and understanding patient preferences in medical institutions.

The proposal unit can estimate the user's emotions and adjust the manner of presenting proposals based on the estimated emotions. The proposal unit, for example, estimates the user's emotions and adjusts the manner of presenting proposals based on the estimated emotions. For example, if the user is feeling stressed, the proposal unit proposes meal plans containing ingredients with relaxing effects. If the user is relaxed, the proposal unit proposes trying new recipes and ingredients. Furthermore, if the user is tired, the proposal unit can propose meal plans suitable for energy replenishment. Thus, the proposal unit can provide more appropriate proposals by adjusting the manner of presenting proposals according to the user's emotions. Emotion estimation is realized using, for example, an emotion engine or generative AI with emotion estimation functions. Generative AI may be text generation AI (e.g., LLM) or multimodal generative AI, but is not limited to these examples. Some or all of the above-described processing in the proposal unit may be performed using AI or without using AI. For example, the proposal unit can use an AI model that receives user emotion data as input and adjusts the manner of presenting proposals. Specifically, the proposal unit receives multimodal data for emotion estimation, such as audio data (e.g., conversation waveform data, sampling rate 16 kHz, 1-minute audio clips), text data (e.g., chat history, SNS posts, up to 512 tokens per post), and image data (e.g., facial expression images, resolution 224×224 pixels, RGB 3 channels) as input. The proposal unit performs preprocessing such as normalization (e.g., Mel spectrogram conversion for audio, tokenization for text, resizing and standardization for images) and feature extraction (e.g., audio emotion features, BERT embeddings, facial expression feature extraction using CNN) before inputting the data into a Transformer-based multimodal emotion estimation model. The AI model integrates the input multidimensional tensors (e.g., 128-dimensional audio feature vector, 768-dimensional text embedding, 512-dimensional image feature vector) and generates output such as emotion labels (e.g., stress, relaxation, fatigue, etc.), emotion intensity scores (e.g., 0.0-1.0), and estimation confidence (e.g., probability distribution). Output examples include “emotion=stress, intensity=0.80” or “emotion=relaxation, intensity=0.60.” Based on these emotion estimation results, the proposal unit applies a proposal expression adjustment algorithm (e.g., if stress intensity is high, emphasize gentle tone and encouraging expressions; if relaxation intensity is high, emphasize novelty and challenging expressions; if fatigue intensity is high, select concise and easy-to-understand expressions) and passes parameters to the meal plan proposal sentence generation module. For example, for the same “grilled salmon+vegetable soup” proposal, during stress, the expression may be “A gentle-tasting menu to soothe the fatigue of the day”; during relaxation, “Would you like to try a new arrangement of grilled salmon?”; and during fatigue, “Quick to prepare and ideal for energy replenishment.” The AI model incorporates penalty terms such as “user emotion fit,” “expression diversity,” and “user satisfaction” into the loss function and updates weights using gradient descent or Adam optimization. Furthermore, the proposal unit sequentially learns user feedback (e.g., satisfaction with proposal expressions, comprehension, implementation status, etc.) and can personalize the model using online learning functions. As a technical effect, the proposal unit can automate real-time adjustment of expressions according to the user's psychological state, thereby delivering remarkable effects such as improved proposal acceptance rate, increased user satisfaction, and improved system operation efficiency. Specific application fields include personal health management apps, patient guidance in medical institutions, user support in nursing care facilities, corporate welfare systems, and mental care support for sports teams.

The proposal unit can adjust the level of detail of proposals based on the importance of the meal plan at the time of proposal. The proposal unit, for example, adjusts the level of detail of proposals based on the importance of the meal plan at the time of proposal. For example, if the user is on a diet, the proposal unit proposes meal plans including detailed information on calories and nutrients. If the user has specific health goals, the proposal unit can propose detailed meal plans tailored to those goals. Furthermore, if the user has specific allergies, the proposal unit can propose detailed meal plans that do not contain those allergens. Thus, the proposal unit can provide more appropriate proposals by adjusting the level of detail of proposals based on the importance of the meal plan. Some or all of the above-described processing in the proposal unit may be performed using AI or without using AI. For example, the proposal unit can use an AI model that receives meal plan importance data as input and adjusts the level of detail of proposals. Specifically, the proposal unit stores user health goals, diet status, allergy information, doctor's instructions, and past meal history as multidimensional vectors (e.g., 128 dimensions including target weight, target period, medical history, allergen list, meal history vector, etc.) in a database and performs normalization, categorization, and quantification before inputting the data into Transformer-based large language models or multimodal models. The AI model receives input vectors (e.g., target weight=60 kg, target period=3 months, allergy=wheat, health goal=blood sugar control, etc.) and generates output such as “proposal detail level parameters (e.g., detail level=high, display calories, nutrients, cooking procedure, ingredient origin; detail level=medium, display only calories and nutrients; detail level=low, display only menu name and brief description).” Output examples include “detail level=high, breakfast=oatmeal+yogurt (calories: 250 kcal, protein: 10 g, fat: 5 g, carbohydrates: 40 g, cooking procedure: 3 steps, recommended recipe URL)” or “detail level=low, breakfast=natto rice.” The proposal unit incorporates penalty terms such as “user goal fit,” “information overload avoidance,” and “user satisfaction” into the loss function of the AI model and updates weights using gradient descent or Adam optimization. Furthermore, the proposal unit sequentially learns user feedback (e.g., satisfaction with detail level, comprehension, implementation status, etc.) and can personalize the model using online learning functions. As a technical effect, the proposal unit can automatically adjust the optimal amount of information in real time according to the user's situation and objectives, thereby preventing confusion due to information overload or dissatisfaction due to lack of information, and delivering remarkable effects such as improved proposal acceptance rate, health management accuracy, user satisfaction, and system operation efficiency. Specific application fields include personal health management apps, patient guidance in medical institutions, corporate health management support, meal management in nursing care facilities, and nutrition guidance for sports teams.

The proposal unit can apply different proposal algorithms according to the category of the meal plan at the time of proposal. The proposal unit, for example, applies different proposal algorithms according to the category of the meal plan at the time of proposal. For example, for diet meals, the proposal unit applies an algorithm that proposes low-calorie and nutritionally balanced meal plans. For meals for athletes, the proposal unit can apply an algorithm that proposes meal plans containing nutrients necessary for training. Furthermore, for allergy-friendly meals, the proposal unit can apply an algorithm that proposes meal plans that do not contain allergens. Thus, the proposal unit can provide more appropriate proposals by applying different proposal algorithms according to the category of the meal plan. Some or all of the above-described processing in the proposal unit may be performed using AI or without using AI. For example, the proposal unit can use an AI model that receives meal plan category data as input and applies different proposal algorithms. Specifically, the proposal unit inputs user objectives and situations (e.g., diet, sports, allergy-friendly, health maintenance, muscle building, etc.) as category labels and selectively applies different AI sub-models or algorithm modules for each category. For example, for the diet category, a Transformer-based model with a loss function emphasizing calorie constraints, nutritional balance, and satiety index is used; for the sports category, a model with a loss function emphasizing target intake of protein, carbohydrates, and vitamins is used; and for the allergy-friendly category, a model with strong penalty terms for allergen exclusion is applied. Input examples include “category=diet, age=35, gender=female, target weight=60 kg,” “category=sports, sport type=soccer, training frequency=five times a week,” or “category=allergy, allergens=egg, wheat.” Output examples include “for diet: breakfast=oatmeal+yogurt, lunch=chicken breast salad, dinner=grilled salmon+vegetable soup; for sports: breakfast=high-protein omelet+whole grain bread, lunch=grilled chicken and vegetables, dinner=braised fish+brown rice; for allergy-friendly: breakfast=natto rice (egg and wheat-free), lunch=grilled mackerel, dinner=miso soup with plenty of vegetables.” The proposal unit combines different loss functions, optimization algorithms, and data augmentation methods for each category, thereby achieving high-precision optimization specialized for each category, which is different from conventional uniform proposals by a single model. Furthermore, the proposal unit sequentially learns user feedback and category change history and can personalize the model using online learning functions. As a technical effect, the proposal unit can automate optimal algorithm selection according to the user's objectives and situation, thereby delivering remarkable effects such as improved proposal accuracy, diversity, user satisfaction, and system operation efficiency. Specific application fields include personal health management apps, patient guidance in medical institutions, nutrition management for sports teams, meal optimization in nursing care facilities, and corporate welfare systems.

The proposal unit can estimate the user's emotions and adjust the length of the proposal based on the estimated emotions. The proposal unit, for example, estimates the user's emotions and adjusts the length of the proposal based on the estimated emotions. For example, if the user is feeling stressed, the proposal unit provides concise and focused proposals. If the user is relaxed, the proposal unit can provide proposals with detailed explanations. Furthermore, if the user is in a hurry, the proposal unit can provide proposals that can be understood in a short time. Thus, the proposal unit can provide more appropriate proposals by adjusting the length of the proposal according to the user's emotions. Emotion estimation is realized using, for example, an emotion engine or generative AI with emotion estimation functions. Generative AI may be text generation AI (e.g., LLM) or multimodal generative AI, but is not limited to these examples. Some or all of the above-described processing in the proposal unit may be performed using AI or without using AI. For example, the proposal unit can use an AI model that receives user emotion data as input and adjusts the length of the proposal. Specifically, the proposal unit receives multimodal data for emotion estimation, such as audio data (e.g., conversation waveform data, sampling rate 16 kHz), text data (e.g., chat history, SNS posts, up to 512 tokens per post), and image data (e.g., facial expression images, resolution 224×224 pixels) as input. The proposal unit performs preprocessing and feature extraction (e.g., audio emotion features, BERT embeddings, facial expression feature extraction using CNN) before inputting the data into a Transformer-based multimodal emotion estimation model. The AI model integrates the input multidimensional tensors (e.g., 128-dimensional audio feature vector, 768-dimensional text embedding, 512-dimensional image feature vector) and generates output such as emotion labels (e.g., stress, relaxation, fatigue, etc.), emotion intensity scores (e.g., 0.0-1.0), and estimation confidence (e.g., probability distribution). Output examples include “emotion=stress, intensity=0.80” or “emotion=relaxation, intensity=0.60.” Based on these emotion estimation results, the proposal unit applies a proposal length adjustment algorithm (e.g., if stress intensity is high, extract only key points and propose in 1-2 sentences; if relaxation intensity is high, include detailed explanations and background information and propose in 5 or more sentences; if fatigue or urgency is high, select the shortest expression) and passes parameters to the meal plan proposal sentence generation module. For example, for the same “grilled salmon+vegetable soup” proposal, during stress, the expression may be “Grilled salmon and vegetable soup are recommended”; during relaxation, “Grilled salmon is rich in omega-3 fatty acids, and combining it with vegetable soup improves nutritional balance. The cooking is easy, so please give it a try,” and so on, with differences in length automatically generated. The AI model incorporates penalty terms such as “user emotion fit,” “expression length optimization,” and “user satisfaction” into the loss function and updates weights using gradient descent or Adam optimization. Furthermore, the proposal unit sequentially learns user feedback (e.g., satisfaction with proposal length, comprehension, implementation status, etc.) and can personalize the model using online learning functions. As a technical effect, the proposal unit can automatically adjust the optimal amount of information in real time according to the user's psychological state and situation, thereby preventing stress due to information overload or lack of information, and delivering remarkable effects such as improved proposal acceptance rate, user satisfaction, and system operation efficiency. Specific application fields include personal health management apps, patient guidance in medical institutions, user support in nursing care facilities, corporate welfare systems, and mental care support for sports teams.

The proposal unit can determine the priority of proposals based on the submission timing of the meal plan at the time of proposal. The proposal unit, for example, determines the priority of proposals based on the submission timing of the meal plan at the time of proposal. For example, if the user requests a breakfast proposal, the proposal unit prioritizes proposing meal plans suitable for breakfast. If the user requests a dinner proposal, the proposal unit can prioritize proposing meal plans suitable for dinner. Furthermore, if the user requests a proposal for a specific event (e.g., party), the proposal unit can prioritize proposing meal plans suitable for that event. Thus, the proposal unit can provide more appropriate proposals by determining the priority of proposals based on the submission timing of the meal plan. Some or all of the above-described processing in the proposal unit may be performed using AI or without using AI. For example, the proposal unit can use an AI model that receives meal plan submission timing data as input and determines the priority of proposals. Specifically, the proposal unit stores submission timing data obtained from the user (e.g., desired meal time, event date and time, meal category (breakfast, lunch, dinner, snack), event type (birthday, meeting, sports tournament, etc.)) as multidimensional vectors (e.g., time information numerically expressed in 24-hour format, event type one-hot encoded, meal category categorized) in a database and performs normalization (e.g., scaling time to 0-1), categorization, and quantification before inputting the data into Transformer-based large language models or multimodal models. The AI model receives input vectors (e.g., desired time=7:30, meal category=breakfast, event type=normal; desired time=19:00, meal category=dinner, event type=party, etc.) and generates output such as “meal plan list with proposal priority scores (e.g., breakfast plan priority 0.95, dinner plan priority 0.90, party plan priority 0.99).” Output examples include “priority=0.98, breakfast=oatmeal+yogurt,” “priority=0.95, dinner=grilled salmon+vegetable soup,” “priority=0.99, party=roast beef+salad+dessert.” The proposal unit incorporates penalty terms such as “submission timing fit,” “event fit,” and “user satisfaction” into the loss function of the AI model and updates weights using gradient descent or Adam optimization. Furthermore, the proposal unit sequentially learns user feedback (e.g., suitability of proposal timing, satisfaction with event proposals, implementation status, etc.) and can personalize the model using online learning functions. As a technical effect, the proposal unit can automate real-time proposal of meal plans at optimal timing according to the user's lifestyle rhythm and event schedule, thereby delivering remarkable effects such as improved proposal acceptance rate, increased user satisfaction, improved health management accuracy, and improved system operation efficiency. Specific application fields include personal health management apps, corporate welfare systems, patient meal guidance in medical institutions, event meal management for sports teams, and meal scheduling in nursing care facilities.

The proposal unit can adjust the order of proposals based on the relevance of the meal plan at the time of proposal. The proposal unit, for example, adjusts the order of proposals based on the relevance of the meal plan at the time of proposal. For example, if the user is on a diet, the proposal unit first proposes low-calorie and nutritionally balanced meal plans. If the user has specific allergies, the proposal unit can first propose meal plans that do not contain those allergens. Furthermore, if the user needs specific nutrients, the proposal unit can first propose meal plans containing those nutrients. Thus, the proposal unit can provide more appropriate proposals by adjusting the order of proposals based on the relevance of the meal plan. Some or all of the above-described processing in the proposal unit may be performed using AI or without using AI. For example, the proposal unit can use an AI model that receives meal plan relevance data as input and adjusts the order of proposals. Specifically, the proposal unit stores user health goals (e.g., diet, muscle building, health maintenance), allergy information (e.g., egg, wheat, dairy, etc.), required nutrients (e.g., iron, protein, vitamin D, etc.), preferences, and past meal history as multidimensional vectors (e.g., 128 dimensions) in a database and performs normalization, categorization, and quantification before inputting the data into Transformer-based large language models or multimodal models. The AI model receives input vectors (e.g., goal=diet, allergy=egg, required nutrient=iron, preference=Japanese cuisine, etc.) and generates output such as “meal plan list with relevance scores (e.g., plan A=0.98, plan B=0.92, plan C=0.85)” and arranges proposals in order of relevance. Output examples include “relevance=0.98, breakfast=natto rice (low-calorie, egg-free, iron-fortified),” “relevance=0.92, lunch=chicken breast salad,” “relevance=0.85, dinner=grilled salmon+vegetable soup.” The proposal unit incorporates penalty terms such as “relevance maximization,” “allergen avoidance,” “nutrient sufficiency,” and “user satisfaction” into the loss function of the AI model and updates weights using gradient descent or Adam optimization. Furthermore, the proposal unit sequentially learns user feedback (e.g., satisfaction with proposal order, implementation status, satisfaction, etc.) and can personalize the model using online learning functions. As a technical effect, the proposal unit can automate real-time proposal of meal plans in optimal order according to the user's situation and needs, thereby delivering remarkable effects such as improved proposal acceptance rate, improved health management accuracy, increased user satisfaction, and improved system operation efficiency. Specific application fields include personal health management apps, patient guidance in medical institutions, nutrition management for sports teams, meal optimization in nursing care facilities, and corporate welfare systems.

The reception unit can estimate the user's emotions and adjust the feedback reception method based on the estimated emotions. For example, the reception unit estimates the user's emotions and adjusts the feedback reception method according to the estimated emotions. For instance, if the user is feeling stressed, the reception unit provides a concise and quick feedback method. If the user is relaxed, the reception unit can provide a method that requests detailed feedback. Furthermore, if the user is in a hurry, the reception unit can also provide a feedback method using voice input. By adjusting the feedback reception method according to the user's emotions, the reception unit can receive more appropriate feedback. Emotion estimation is realized, for example, by using an emotion estimation function such as an emotion engine or generative AI. Generative AI may include, for example, text generation AI (such as LLM) or multimodal generative AI, but is not limited to these examples. Some or all of the above-described processing in the reception unit may be performed using AI or without using AI. For example, the reception unit can adjust the feedback reception method by using an AI model that takes the user's emotion data as input and adjusts the feedback reception method. Specifically, the reception unit receives multimodal data such as voice data (e.g., waveform data of conversational speech, sampling rate 16 kHz, 1-minute audio clip), text data (e.g., chat history, SNS posts, up to 512 tokens per post), and image data (e.g., facial expression images, resolution 224×224 pixels, RGB 3 channels) as input for emotion estimation. The reception unit applies preprocessing such as normalization (e.g., Mel spectrogram conversion for audio, tokenization for text, resizing and standardization for images) and feature extraction (e.g., audio emotion features, BERT embeddings, facial expression feature extraction by CNN), and inputs the processed data into a Transformer-based multimodal emotion estimation model. The AI model integrates the input multidimensional tensors (e.g., 128-dimensional audio feature vector, 768-dimensional text embedding, 512-dimensional image feature vector) and outputs emotion labels (e.g., stress, relaxation, fatigue, etc.), emotion intensity scores (e.g., 0.0-1.0), and estimation confidence (e.g., probability distribution). Examples of output include “emotion=stress, intensity=0.80” and “emotion=relaxation, intensity=0.60”. Based on these emotion estimation results, the reception unit applies a feedback reception method adjustment algorithm (e.g., if stress intensity is high, a simple UI with selection or one-tap options; if relaxation intensity is high, free text or detailed questionnaire; if in a hurry, automatic presentation of voice input UI) and passes parameters to the user interface generation module. The AI model incorporates penalty terms such as “user emotion compatibility”, “reception efficiency”, and “user satisfaction” into the loss function, and updates weights using gradient descent or Adam optimization. Furthermore, the reception unit can sequentially learn user feedback (e.g., satisfaction with the reception method, input burden, implementation rate, etc.) and personalize the model through online learning. As a technical effect, the reception unit can automatically adjust the optimal feedback reception method in real time according to the user's psychological state and situation, thereby achieving remarkable effects such as improved feedback collection rate, user satisfaction, and system operational efficiency. Specific application fields include personal health management apps, patient guidance in medical institutions, user support in nursing care facilities, corporate welfare systems, and mental care support for sports teams.

The reception unit can refer to the user's past feedback history at the time of feedback reception and select the optimal reception method. For example, the reception unit refers to the user's past feedback history at the time of feedback reception to select the optimal reception method. For instance, the reception unit preferentially provides feedback methods (e.g., text, voice) that the user has used in the past. Additionally, the reception unit can provide feedback methods suitable for specific time periods based on the user's past feedback history. Furthermore, the reception unit can analyze the user's past feedback content and automatically generate related questions. By referring to the user's past feedback history, the reception unit can select a more appropriate feedback reception method. Some or all of the above-described processing in the reception unit may be performed using AI or without using AI. For example, the reception unit can perform feedback reception using an AI model that takes the user's past feedback history data as input and selects the optimal reception method. Specifically, the reception unit stores the user's past feedback history data (e.g., reception method [text, voice, image], reception time, content detail level, response time, satisfaction score, Q&A history, etc.) as a time-series database and inputs each history as a multidimensional vector (e.g., 32 dimensions per instance, 30×32-dimensional tensor for one month) into the AI model. The reception unit applies preprocessing such as normalization (e.g., scaling reception time to 0-1), categorization (e.g., one-hot encoding for reception method), and quantification (e.g., converting satisfaction score to real values). The AI model may be a Transformer-based time-series analysis model or an LSTM-type recurrent neural network. Examples of input include “2024/6/1: method=text, time=20:00, satisfaction=4/5” and “2024/6/2: method=voice, time=8:00, satisfaction=5/5”. The AI model analyzes these history tensors and outputs structured data such as “recommended reception method (e.g., voice input, text input, image upload, etc.)”, “recommended reception timing (e.g., morning, night, after meals, etc.)”, and “related question list (e.g., additional questions based on previous dissatisfaction)”. Examples of output include “recommended method=voice input, timing=morning, question=‘How was the seasoning?’” and “recommended method=text input, timing=night, question=‘Was the cooking time appropriate?’”. The reception unit incorporates penalty terms such as “reception efficiency”, “user burden reduction”, and “maximization of satisfaction” into the AI model's loss function, and updates weights using gradient descent or Adam optimization. Furthermore, the reception unit can sequentially learn new user feedback and reactions to reception methods, and personalize the model through online learning. As a technical effect, the reception unit can generate feedback reception designs optimized for each user in real time, thereby achieving remarkable effects such as improved feedback collection rate, user satisfaction, and system operational efficiency. Specific application fields include personal health management apps, patient guidance in medical institutions, user support in nursing care facilities, corporate welfare systems, and mental care support for sports teams.

The reception unit can estimate the user's emotions and determine the priority of feedback based on the estimated emotions. For example, the reception unit estimates the user's emotions and determines the priority of feedback according to the estimated emotions. For instance, if the user is feeling stressed, the reception unit prioritizes feedback that requires prompt response. If the user is relaxed, the reception unit can prioritize detailed feedback. Furthermore, if the user is in a hurry, the reception unit can also prioritize concise feedback. By determining the priority of feedback according to the user's emotions, the reception unit can process feedback more appropriately. Emotion estimation is realized, for example, by using an emotion estimation function such as an emotion engine or generative AI. Generative AI may include, for example, text generation AI (such as LLM) or multimodal generative AI, but is not limited to these examples. Some or all of the above-described processing in the reception unit may be performed using AI or without using AI. For example, the reception unit can process feedback by using an AI model that takes the user's emotion data as input and determines the priority of feedback. Specifically, the reception unit receives multimodal data such as voice data (e.g., waveform data of conversational speech, sampling rate 16 kHz, 1-minute audio clip), text data (e.g., chat history, SNS posts, up to 512 tokens per post), and image data (e.g., facial expression images, resolution 224×224 pixels, RGB 3 channels) as input for emotion estimation. The reception unit applies preprocessing such as normalization (e.g., Mel spectrogram conversion for audio, tokenization for text, resizing and standardization for images) and feature extraction (e.g., audio emotion features, BERT embeddings, facial expression feature extraction by CNN), and inputs the processed data into a Transformer-based multimodal emotion estimation model. The AI model integrates the input multidimensional tensors (e.g., 128-dimensional audio feature vector, 768-dimensional text embedding, 512-dimensional image feature vector) and outputs emotion labels (e.g., stress, relaxation, fatigue, etc.), emotion intensity scores (e.g., 0.0-1.0), and estimation confidence (e.g., probability distribution). Examples of output include “emotion=stress, intensity=0.80” and “emotion=relaxation, intensity=0.60”. Based on these emotion estimation results, the reception unit applies a feedback priority determination algorithm (e.g., if stress intensity is high, prioritize feedback with urgency flags; if relaxation intensity is high, prioritize detailed feedback; if in a hurry, prioritize short or selection-type feedback) and passes parameters to the feedback processing module. The AI model incorporates penalty terms such as “user emotion compatibility”, “processing efficiency”, and “user satisfaction” into the loss function, and updates weights using gradient descent or Adam optimization. Furthermore, the reception unit can sequentially learn user feedback (e.g., acceptance of priority, processing speed, satisfaction, etc.) and personalize the model through online learning. As a technical effect, the reception unit can automate the optimal feedback priority determination in real time according to the user's psychological state and situation, thereby achieving remarkable effects such as improved feedback processing efficiency, user satisfaction, and system operational efficiency. Specific application fields include personal health management apps, patient guidance in medical institutions, user support in nursing care facilities, corporate welfare systems, and mental care support for sports teams.

The reception unit can select the optimal reception method by considering the user's device information at the time of feedback reception. For example, the reception unit considers the user's device information at the time of feedback reception to select the optimal reception method. For instance, if the user is using a smartphone, the reception unit provides a feedback method using voice input. If the user is using a tablet, the reception unit can provide a feedback method optimized for a large screen. Furthermore, if the user is using a personal computer, the reception unit can also provide a feedback method using keyboard input. By considering the user's device information, the reception unit can select a more appropriate feedback reception method. Some or all of the above-described processing in the reception unit may be performed using AI or without using AI. For example, the reception unit can perform feedback reception by using an AI model that takes the user's device information as input and selects the optimal reception method. Specifically, the reception unit stores the user's device information (e.g., device type [smartphone, tablet, PC], OS version, screen size, input interface [touch, keyboard, voice], usage environment [home, outside, in transit, etc.]) as a multidimensional vector (e.g., one-hot encoding for device type, numerical value for screen size, categorization for input interface) in a database, applies preprocessing such as normalization, categorization, and quantification, and inputs the data into a Transformer-based large language model or multimodal model. The AI model receives the input vector (e.g., device=smartphone, screen size=5.5 inches, input=voice, usage environment=outside, etc.) and outputs “recommended reception method (e.g., voice input UI, touch-optimized UI, keyboard input UI, etc.)”. Examples of output include “recommended method=voice input UI (for smartphone)”, “recommended method=touch-optimized UI (for tablet)”, and “recommended method=keyboard input UI (for PC)”. The reception unit incorporates penalty terms such as “device compatibility”, “input efficiency”, and “user satisfaction” into the AI model's loss function, and updates weights using gradient descent or Adam optimization. Furthermore, the reception unit can sequentially learn user feedback (e.g., satisfaction with the reception method, input burden, implementation rate, etc.) and personalize the model through online learning. As a technical effect, the reception unit can automatically adjust the optimal feedback reception method in real time according to the user's device and environment, thereby achieving remarkable effects such as improved feedback collection rate, user satisfaction, and system operational efficiency. Specific application fields include personal health management apps, patient guidance in medical institutions, user support in nursing care facilities, corporate welfare systems, and mental care support for sports teams.

The system according to the embodiment is not limited to the above examples, and various modifications are possible as described below. Specifically, the system can replace the AI model architecture from a Transformer-based large language model to other neural network structures such as convolutional neural networks, recurrent neural networks, or graph neural networks. The database configuration can also adopt various data storage methods such as relational databases, NoSQL databases, time-series databases, or graph databases. Furthermore, communication methods between modules for information collection, proposal, and feedback processing can utilize REST API, gRPC, message queues, event-driven architectures, and so on. The learning methods for AI models can also apply various techniques such as batch learning, online learning, transfer learning, reinforcement learning, or self-supervised learning. In addition, the user interface can be implemented in various forms such as web applications, mobile applications, voice dialogue systems, chatbots, or smart speaker integration. Moreover, to enhance security, technologies such as data encryption, access control, anonymization processing, and audit log recording can be combined. These variations enable the system to flexibly change its configuration according to the usage environment and user requirements, thereby achieving technical effects such as improved scalability, maintainability, operational efficiency, and security. Specific application fields include personal health management apps, corporate welfare systems, nutritional guidance support in medical institutions, meal management for sports teams, meal optimization in nursing care facilities, health promotion projects by local governments, and school meal management systems.

The proposal unit can estimate the user's emotions and adjust the content of the meal plan proposal based on the estimated emotions. For example, if the user is feeling stressed, the proposal unit proposes a meal plan that includes ingredients with relaxing effects. If the user is relaxed, the proposal unit can propose trying new recipes or ingredients. Furthermore, if the user is fatigued, the proposal unit can also propose a meal plan suitable for energy replenishment. By adjusting the proposal content according to the user's emotions, the proposal unit can make more appropriate proposals. Emotion estimation is realized, for example, by using an emotion estimation function such as an emotion engine or generative AI. Generative AI may include, for example, text generation AI (such as LLM) or multimodal generative AI, but is not limited to these examples. Some or all of the above-described processing in the proposal unit may be performed using AI or without using AI. For example, the proposal unit can adjust the proposal content by using an AI model that takes the user's emotion data as input and adjusts the proposal content. Specifically, the proposal unit receives multimodal data such as voice data (e.g., waveform data of conversational speech, sampling rate 16 kHz, 1-minute audio clip), text data (e.g., chat history, SNS posts, up to 512 tokens per post), and image data (e.g., facial expression images, resolution 224×224 pixels, RGB 3 channels) as input for emotion estimation. The proposal unit applies preprocessing such as normalization (e.g., Mel spectrogram conversion for audio, tokenization for text, resizing and standardization for images) and feature extraction (e.g., audio emotion features, BERT embeddings, facial expression feature extraction by CNN), and inputs the processed data into a Transformer-based multimodal emotion estimation model. The AI model integrates the input multidimensional tensors (e.g., 128-dimensional audio feature vector, 768-dimensional text embedding, 512-dimensional image feature vector) and outputs emotion labels (e.g., stress, relaxation, fatigue, etc.), emotion intensity scores (e.g., 0.0-1.0), and estimation confidence (e.g., probability distribution). Examples of output include “emotion=stress, intensity=0.80” and “emotion=relaxation, intensity=0.60”. Based on these emotion estimation results, the proposal unit applies a proposal content adjustment algorithm (e.g., if stress intensity is high, prioritize plans including ingredients with high relaxing effects [e.g., herbal tea, salmon, banana, etc.]; if relaxation intensity is high, propose novel or challenging recipes [e.g., ethnic cuisine, seasonal menus, etc.]; if fatigue intensity is high, prioritize plans including high-carbohydrate and high-protein ingredients suitable for energy replenishment [e.g., brown rice, chicken breast, beans, etc.]) and passes parameters to the meal plan generation module. The AI model incorporates penalty terms such as “user emotion compatibility”, “proposal content diversity”, and “user satisfaction” into the loss function, and updates weights using gradient descent or Adam optimization. Furthermore, the proposal unit can sequentially learn user feedback (e.g., satisfaction with proposal content, feasibility, comprehension, etc.) and personalize the model through online learning. As a technical effect, the proposal unit can automatically adjust the optimal meal plan content in real time according to the user's psychological state and situation, thereby achieving remarkable effects such as improved proposal acceptance rate, user satisfaction, health management accuracy, and system operational efficiency. Specific application fields include personal health management apps, patient guidance in medical institutions, user support in nursing care facilities, corporate welfare systems, and mental care support for sports teams.

The collection unit can analyze the user's past meal history and select the optimal information collection method. For example, the collection unit prioritizes the collection of related information based on dishes the user has enjoyed in the past. The collection unit can also collect information considering allergy information based on ingredients the user has avoided in the past. Furthermore, if a specific nutrient is found to be lacking from the user's meal history, the collection unit can collect information on ingredients containing that nutrient. By analyzing the user's past meal history, the collection unit can perform more appropriate information collection. Some or all of the above-described processing in the collection unit may be performed using AI or without using AI. For example, the collection unit can select the information collection method by using an AI model that takes the user's past meal history data as input and selects the optimal information collection method. Specifically, the collection unit stores the user's past meal history data (e.g., date, meal content, calorie intake, nutrient distribution, ingredient list, intake amount, meal satisfaction score, etc.) as a time-series database and inputs each meal history as a multidimensional vector (e.g., 64 dimensions per meal, 7×64-dimensional tensor for one week) into the AI model. The collection unit applies preprocessing such as normalization (e.g., scaling calories to 0-1 per meal), categorization (e.g., one-hot encoding for cuisine genre), and quantification (e.g., converting nutrient intake to real-valued vectors). The AI model may be a Transformer-based time-series analysis model or an LSTM-type recurrent neural network. Examples of input include “2024/6/1: breakfast=natto rice, lunch=chicken breast salad, dinner=grilled salmon+vegetable soup, calories, protein, fat, carbohydrate, vitamin amount for each meal” and “2024/6/2: breakfast=oatmeal+yogurt, lunch=grilled chicken and vegetables, dinner=simmered fish+brown rice”. The AI model analyzes these history tensors and outputs structured data such as “information categories to be prioritized for collection (e.g., Japanese recipes, low-carb ingredients, high-protein ingredients, allergen-free recipes, etc.)”, “recommended collection methods (e.g., questionnaire format, image upload, voice input, etc.)”, and “collection frequency (e.g., once a week, after every meal)”. Examples of output include “priority category=high-protein Japanese cuisine, recommended method=image+text, frequency=after every meal” and “priority category=low-carb Italian, recommended method=questionnaire, frequency=twice a week”. By incorporating penalty terms such as “information collection efficiency”, “user burden reduction”, “allergen avoidance”, and “nutrient sufficiency” into the AI model's loss function, the collection unit achieves optimization in high-dimensional space, unlike conventional simple history reference or human heuristics for collection design. Furthermore, the collection unit can sequentially learn user feedback and new meal history, and personalize the model through online learning. As a technical effect, the collection unit can generate information collection designs optimized for each user in real time, thereby achieving remarkable effects such as improved data quality, user satisfaction, and reduced system operational costs. Specific application fields include personal health management apps, nutritional guidance support in medical institutions, meal management for sports teams, and meal history monitoring in nursing care facilities.

The proposal unit can estimate the user's emotions and adjust the manner of presenting proposals based on the estimated emotions. For example, if the user is feeling stressed, the proposal unit proposes a meal plan that includes ingredients with relaxing effects. If the user is relaxed, the proposal unit can propose trying new recipes or ingredients. Furthermore, if the user is fatigued, the proposal unit can also propose a meal plan suitable for energy replenishment. By adjusting the manner of presenting proposals according to the user's emotions, the proposal unit can make more appropriate proposals. Emotion estimation is realized, for example, by using an emotion estimation function such as an emotion engine or generative AI. Generative AI may include, for example, text generation AI (such as LLM) or multimodal generative AI, but is not limited to these examples. Some or all of the above-described processing in the proposal unit may be performed using AI or without using AI. For example, the proposal unit can adjust the manner of presenting proposals by using an AI model that takes the user's emotion data as input and adjusts the manner of presenting proposals. Specifically, the proposal unit receives multimodal data such as voice data (e.g., waveform data of conversational speech, sampling rate 16 kHz, 1-minute audio clip), text data (e.g., chat history, SNS posts, up to 512 tokens per post), and image data (e.g., facial expression images, resolution 224×224 pixels, RGB 3 channels) as input for emotion estimation. The proposal unit applies preprocessing such as normalization (e.g., Mel spectrogram conversion for audio, tokenization for text, resizing and standardization for images) and feature extraction (e.g., audio emotion features, BERT embeddings, facial expression feature extraction by CNN), and inputs the processed data into a Transformer-based multimodal emotion estimation model. The AI model integrates the input multidimensional tensors (e.g., 128-dimensional audio feature vector, 768-dimensional text embedding, 512-dimensional image feature vector) and outputs emotion labels (e.g., stress, relaxation, fatigue, etc.), emotion intensity scores (e.g., 0.0-1.0), and estimation confidence (e.g., probability distribution). Examples of output include “emotion=stress, intensity=0.80” and “emotion=relaxation, intensity=0.60”. Based on these emotion estimation results, the proposal unit applies a proposal expression adjustment algorithm (e.g., if stress intensity is high, emphasize gentle tone and encouraging expressions; if relaxation intensity is high, emphasize novelty and challenging expressions; if fatigue intensity is high, select concise and easy-to-understand expressions) and passes parameters to the meal plan proposal text generation module. For example, for the same “grilled salmon+vegetable soup” proposal, the system automatically generates different expressions such as “A gentle-tasting menu to soothe the fatigue of the day” for stress, “Would you like to try a new arrangement of grilled salmon?” for relaxation, and “Quick to prepare and ideal for energy replenishment” for fatigue. The AI model incorporates penalty terms such as “user emotion compatibility”, “expression diversity”, and “user satisfaction” into the loss function, and updates weights using gradient descent or Adam optimization. Furthermore, the proposal unit can sequentially learn user feedback (e.g., satisfaction with proposal expressions, comprehension, feasibility, etc.) and personalize the model through online learning. As a technical effect, the proposal unit can automate expression adjustment in real time according to the user's psychological state, thereby achieving remarkable effects such as improved proposal acceptance rate, user satisfaction, and system operational efficiency. Specific application fields include personal health management apps, patient guidance in medical institutions, user support in nursing care facilities, corporate welfare systems, and mental care support for sports teams.

The collection unit can prioritize the collection of highly relevant information based on the user's geographic location information at the time of information collection. For example, the collection unit collects information on local specialties and seasonal ingredients of the user's residential area. If the user is traveling, the collection unit can collect information on the local food culture and recommended restaurants of that area. Furthermore, if the user is planning to move to a specific region, the collection unit can collect information on ingredients and recipes of that region. By considering the user's geographic location information, the collection unit can collect more relevant information. Some or all of the above-described processing in the collection unit may be performed using AI or without using AI. For example, the collection unit can perform information collection by using an AI model that takes the user's geographic location information as input and prioritizes the collection of highly relevant information. Specifically, the collection unit stores the user's geographic location information (e.g., GPS coordinates, prefecture/municipality codes, movement history, length of stay, etc.) as a multidimensional vector (e.g., 16 dimensions including latitude, longitude, region category, movement frequency, etc.) in a database, applies preprocessing such as normalization (e.g., scaling latitude and longitude to 0-1), categorization (e.g., one-hot encoding for region category), and quantification (e.g., converting movement frequency to real values per week). The collection unit inputs these vector data into a Transformer-based large language model or multimodal model. Examples of input include “current location=Chiyoda-ku, Tokyo; movement history=Osaka, Kyoto; length of stay=3 days” and “current location=Sapporo, Hokkaido; movement history=none; length of stay=1 year”. The AI model analyzes these input vectors and outputs structured data such as “information categories to be prioritized for collection (e.g., seasonal vegetables from Hokkaido, local recipe of Sapporo, recommended restaurants in Chiyoda-ku, etc.)” and “recommended collection methods (e.g., local survey, image upload, region-limited recipe search, etc.)”. Examples of output include “priority category=Sapporo local cuisine, recommended method=recipe image collection” and “priority category=Osaka specialties, recommended method=local survey”. By incorporating penalty terms such as “regional relevance”, “information collection efficiency”, and “user satisfaction” into the AI model's loss function, the collection unit achieves optimization in high-dimensional space, unlike conventional simple regional filters or human heuristics for information collection. Furthermore, the collection unit can sequentially learn changes in the user's movement history and regional preferences, and personalize the model through online learning. As a technical effect, the collection unit can optimize information collection in real time according to the user's geographic situation, thereby achieving remarkable effects such as improved information relevance, user satisfaction, and system operational efficiency. Specific application fields include personal health management apps, meal proposal systems for travelers, regional specialty promotion, support for the use of local ingredients in medical institutions, and introduction of regional food culture in nursing care facilities.

The proposal unit can adjust the level of detail of proposals based on the importance of the meal plan at the time of proposal. For example, if the user is on a diet, the proposal unit proposes a meal plan that includes detailed information on calories and nutrients. If the user has specific health goals, the proposal unit can propose a detailed meal plan tailored to those goals. Furthermore, if the user has specific allergies, the proposal unit can also propose a detailed meal plan that does not include those allergens. By adjusting the level of detail of proposals based on the importance of the meal plan, the proposal unit can make more appropriate proposals. Some or all of the above-described processing in the proposal unit may be performed using AI or without using AI. For example, the proposal unit can adjust the level of detail of proposals by using an AI model that takes the meal plan importance data as input and adjusts the level of detail of proposals. Specifically, the proposal unit stores the user's health goals, diet status, allergy information, doctor's instructions, and past meal history as a multidimensional vector (e.g., 128 dimensions including target weight, target period, medical history, allergen list, meal history vector, etc.) in a database, applies normalization, categorization, and quantification, and inputs the data into a Transformer-based large language model or multimodal model. The AI model receives the input vector (e.g., target weight=60 kg, target period=3 months, allergy=wheat, health goal=blood sugar control, etc.) and outputs “proposal detail parameter (e.g., detail=high, display calories, nutrients, cooking steps, ingredient origin; detail=medium, display only calories and nutrients; detail=low, display only menu name and brief description)”. Examples of output include “detail=high, breakfast=oatmeal+yogurt (calories: 250 kcal, protein: 10 g, fat: 5 g, carbohydrates: 40 g, cooking steps: 3, recommended recipe URL)” and “detail=low, breakfast=natto rice”. The proposal unit incorporates penalty terms such as “user goal compatibility”, “information overload avoidance”, and “user satisfaction” into the AI model's loss function, and updates weights using gradient descent or Adam optimization. Furthermore, the proposal unit can sequentially learn user feedback (e.g., satisfaction with detail level, comprehension, feasibility, etc.) and personalize the model through online learning. As a technical effect, the proposal unit can automatically adjust the optimal amount of information according to the user's situation and objectives, thereby preventing confusion due to information overload or dissatisfaction due to lack of information, and achieving remarkable effects such as improved proposal acceptance rate, health management accuracy, user satisfaction, and system operational efficiency. Specific application fields include personal health management apps, patient guidance in medical institutions, corporate health management support, meal management in nursing care facilities, and nutritional guidance for sports teams.

The proposal unit can estimate the user's emotions and adjust the length of proposals based on the estimated emotions. For example, if the user is feeling stressed, the proposal unit provides concise proposals that focus on key points. If the user is relaxed, the proposal unit can provide proposals with detailed explanations. Furthermore, if the user is in a hurry, the proposal unit can also provide proposals that can be understood in a short time. By adjusting the length of proposals according to the user's emotions, the proposal unit can make more appropriate proposals. Emotion estimation is realized, for example, by using an emotion estimation function such as an emotion engine or generative AI. Generative AI may include, for example, text generation AI (such as LLM) or multimodal generative AI, but is not limited to these examples. Some or all of the above-described processing in the proposal unit may be performed using AI or without using AI. For example, the proposal unit can adjust the length of proposals by using an AI model that takes the user's emotion data as input and adjusts the length of proposals. Specifically, the proposal unit receives multimodal data such as voice data (e.g., waveform data of conversational speech, sampling rate 16 kHz), text data (e.g., chat history, SNS posts, up to 512 tokens per post), and image data (e.g., facial expression images, resolution 224×224 pixels) as input for emotion estimation. The proposal unit applies preprocessing and feature extraction (e.g., audio emotion features, BERT embeddings, facial expression feature extraction by CNN), and inputs the processed data into a Transformer-based multimodal emotion estimation model. The AI model integrates the input multidimensional tensors (e.g., 128-dimensional audio feature vector, 768-dimensional text embedding, 512-dimensional image feature vector) and outputs emotion labels (e.g., stress, relaxation, fatigue, etc.), emotion intensity scores (e.g., 0.0-1.0), and estimation confidence (e.g., probability distribution). Examples of output include “emotion=stress, intensity=0.80” and “emotion=relaxation, intensity=0.60”. Based on these emotion estimation results, the proposal unit applies a proposal length adjustment algorithm (e.g., if stress intensity is high, extract only key points and propose in 1-2 sentences; if relaxation intensity is high, include detailed explanations and background information and propose in 5 or more sentences; if fatigued or in a hurry, select the shortest expression) and passes parameters to the meal plan proposal text generation module. For example, for the same “grilled salmon+vegetable soup” proposal, the system automatically generates different lengths such as “Grilled salmon and vegetable soup are recommended” for stress, and “Grilled salmon is rich in omega-3 fatty acids, and combining it with vegetable soup improves nutritional balance. Cooking is easy, so please give it a try” for relaxation. The AI model incorporates penalty terms such as “user emotion compatibility”, “expression length optimization”, and “user satisfaction” into the loss function, and updates weights using gradient descent or Adam optimization. Furthermore, the proposal unit can sequentially learn user feedback (e.g., satisfaction with proposal length, comprehension, feasibility, etc.) and personalize the model through online learning. As a technical effect, the proposal unit can automatically adjust the optimal amount of information in real time according to the user's psychological state and situation, thereby preventing stress due to information overload or lack of information, and achieving remarkable effects such as improved proposal acceptance rate, user satisfaction, and system operational efficiency. Specific application fields include personal health management apps, patient guidance in medical institutions, user support in nursing care facilities, corporate welfare systems, and mental care support for sports teams.

The collection unit can analyze the user's social media activity at the time of information collection and collect relevant information. For example, the collection unit collects information on related recipes and ingredients based on meal photos shared by the user on social media. The collection unit can also analyze posts from cooking accounts followed by the user and collect information that may be of interest. Furthermore, the collection unit can analyze trends in cooking communities the user participates in and collect related information. By analyzing the user's social media activity, the collection unit can collect more relevant information. Some or all of the above-described processing in the collection unit may be performed using AI or without using AI. For example, the collection unit can perform information collection by using an AI model that takes the user's social media data as input and collects relevant information. Specifically, the collection unit stores the user's social media activity data (e.g., post text, images, videos, like history, followed accounts, participating communities, post date and time, etc.) as multidimensional vectors (e.g., 768-dimensional text embedding, 512-dimensional image feature vector, 32-dimensional account category vector, etc.) in a database, applies preprocessing such as normalization (e.g., scaling post frequency to 0-1), categorization (e.g., one-hot encoding for account type), and quantification (e.g., converting number of likes to real values). The collection unit inputs these vector data into a Transformer-based multimodal model. Examples of input include “post text=‘Today's lunch is salmon bowl’, image=salmon bowl photo, followed account=Japanese recipe, post date=2024/6/1” and “post text=‘Trying a new pasta recipe’, image=pasta photo, followed account=Italian cuisine”. The AI model analyzes these input vectors and outputs structured data such as “information categories to be prioritized for collection (e.g., Japanese recipes, salmon dishes, Italian pasta, etc.)” and “recommended collection methods (e.g., image analysis, text mining, community trend analysis, etc.)”. Examples of output include “priority category=salmon recipes, recommended method=image analysis” and “priority category=Italian pasta, recommended method=text mining”. By incorporating penalty terms such as “information relevance”, “user interest compatibility”, and “information collection efficiency” into the AI model's loss function, the collection unit achieves optimization in high-dimensional space, unlike conventional simple keyword searches or human heuristics for information collection. Furthermore, the collection unit can sequentially learn changes in the user's social media activity and new trends, and personalize the model through online learning. As a technical effect, the collection unit can optimize information collection in real time according to the user's interests and preferences, thereby achieving remarkable effects such as improved information relevance, user satisfaction, and system operational efficiency. Specific application fields include personal health management apps, recipe proposal services, meal trend analysis, corporate marketing support, and understanding patient preferences in medical institutions.

The proposal unit can apply different proposal algorithms according to the category of the meal plan at the time of proposal. For example, for diet meals, the proposal unit applies an algorithm that proposes low-calorie and nutritionally balanced meal plans. For meals for athletes, the proposal unit applies an algorithm that proposes meal plans containing nutrients necessary for training. Furthermore, for allergy-friendly meals, the proposal unit can also apply an algorithm that proposes meal plans that do not contain allergens. By applying different proposal algorithms according to the category of the meal plan, the proposal unit can make more appropriate proposals. Some or all of the above-described processing in the proposal unit may be performed using AI or without using AI. For example, the proposal unit can make proposals by using an AI model that takes the meal plan category data as input and applies different proposal algorithms. Specifically, the proposal unit inputs the user's purpose and situation (e.g., diet, sports, allergy-friendly, health maintenance, muscle building, etc.) as category labels, and selectively applies different AI sub-models or algorithm modules for each category. For example, for the diet category, a Transformer-based model with a loss function emphasizing calorie constraints, nutritional balance, and satiety index is used; for the sports category, a model with a loss function emphasizing target intake of protein, carbohydrates, vitamins, etc. is used; and for the allergy-friendly category, a model with strong penalty terms reflecting allergen exclusion constraints is applied. Examples of input include “category=diet, age=35, gender=female, target weight=60 kg”, “category=sports, sport=soccer, training frequency=5 times a week”, and “category=allergy, allergen=egg, wheat”. Examples of output include “for diet: breakfast=oatmeal+yogurt, lunch=chicken breast salad, dinner=grilled salmon+vegetable soup”; “for sports: breakfast=high-protein omelet+whole grain bread, lunch=grilled chicken and vegetables, dinner=simmered fish+brown rice”; and “for allergy-friendly: breakfast=natto rice (egg and wheat-free), lunch=grilled mackerel, dinner=vegetable-rich miso soup”. By combining different loss functions, optimization algorithms, and data augmentation methods for each category, the proposal unit achieves high-precision optimization specialized for each category, unlike conventional uniform proposals by a single model. Furthermore, the proposal unit can sequentially learn user feedback and category change history, and personalize the model through online learning. As a technical effect, the proposal unit can automate optimal algorithm selection according to the user's purpose and situation, thereby achieving remarkable effects such as improved proposal accuracy, diversity, user satisfaction, and system operational efficiency. Specific application fields include personal health management apps, patient guidance in medical institutions, nutrition management for sports teams, meal optimization in nursing care facilities, and corporate welfare systems.

The reception unit can estimate the user's emotions and adjust the feedback reception method based on the estimated emotions. For example, if the user is feeling stressed, the reception unit provides a concise and quick feedback method. If the user is relaxed, the reception unit can provide a method that requests detailed feedback. Furthermore, if the user is in a hurry, the reception unit can also provide a feedback method using voice input. By adjusting the feedback reception method according to the user's emotions, the reception unit can receive more appropriate feedback. Emotion estimation is realized, for example, by using an emotion estimation function such as an emotion engine or generative AI. Generative AI may include, for example, text generation AI (such as LLM) or multimodal generative AI, but is not limited to these examples. Some or all of the above-described processing in the reception unit may be performed using AI or without using AI. For example, the reception unit can adjust the feedback reception method by using an AI model that takes the user's emotion data as input and adjusts the feedback reception method. Specifically, the reception unit receives multimodal data such as voice data (e.g., waveform data of conversational speech, sampling rate 16 kHz, 1-minute audio clip), text data (e.g., chat history, SNS posts, up to 512 tokens per post), and image data (e.g., facial expression images, resolution 224×224 pixels, RGB 3 channels) as input for emotion estimation. The reception unit applies preprocessing such as normalization (e.g., Mel spectrogram conversion for audio, tokenization for text, resizing and standardization for images) and feature extraction (e.g., audio emotion features, BERT embeddings, facial expression feature extraction by CNN), and inputs the processed data into a Transformer-based multimodal emotion estimation model. The AI model integrates the input multidimensional tensors (e.g., 128-dimensional audio feature vector, 768-dimensional text embedding, 512-dimensional image feature vector) and outputs emotion labels (e.g., stress, relaxation, fatigue, etc.), emotion intensity scores (e.g., 0.0-1.0), and estimation confidence (e.g., probability distribution). Examples of output include “emotion=stress, intensity=0.80” and “emotion=relaxation, intensity=0.60”. Based on these emotion estimation results, the reception unit applies a feedback reception method adjustment algorithm (e.g., if stress intensity is high, a simple UI with selection or one-tap options; if relaxation intensity is high, free text or detailed questionnaire; if in a hurry, automatic presentation of voice input UI) and passes parameters to the user interface generation module. The AI model incorporates penalty terms such as “user emotion compatibility”, “reception efficiency”, and “user satisfaction” into the loss function, and updates weights using gradient descent or Adam optimization. Furthermore, the reception unit can sequentially learn user feedback (e.g., satisfaction with the reception method, input burden, implementation rate, etc.) and personalize the model through online learning. As a technical effect, the reception unit can automatically adjust the optimal feedback reception method in real time according to the user's psychological state and situation, thereby achieving remarkable effects such as improved feedback collection rate, user satisfaction, and system operational efficiency. Specific application fields include personal health management apps, patient guidance in medical institutions, user support in nursing care facilities, corporate welfare systems, and mental care support for sports teams.

The proposal unit can determine the priority of proposals based on the submission timing of the meal plan at the time of proposal. For example, if the user requests a breakfast proposal, the proposal unit prioritizes meal plans suitable for breakfast. If the user requests a dinner proposal, the proposal unit can prioritize meal plans suitable for dinner. Furthermore, if the user requests a proposal for a specific event (e.g., party), the proposal unit can also prioritize meal plans suitable for that event. By determining the priority of proposals based on the submission timing of the meal plan, the proposal unit can make more appropriate proposals. Some or all of the above-described processing in the proposal unit may be performed using AI or without using AI. For example, the proposal unit can make proposals by using an AI model that takes the meal plan submission timing data as input and determines the priority of proposals. Specifically, the proposal unit stores submission timing data obtained from the user (e.g., desired meal time, event date and time, meal category [breakfast, lunch, dinner, snack], event type [birthday, meeting, sports tournament, etc.]) as a multidimensional vector (e.g., numerical representation of time information in 24-hour format, one-hot encoding for event type, categorization for meal category) in a database, applies preprocessing such as normalization (e.g., scaling time to 0-1), categorization, and quantification, and inputs the data into a Transformer-based large language model or multimodal model. The AI model receives the input vector (e.g., desired time=7:30, meal category=breakfast, event type=normal; desired time=19:00, meal category=dinner, event type=party, etc.) and outputs “meal plan list with proposal priority scores (e.g., breakfast plan priority 0.95, dinner plan priority 0.90, party plan priority 0.99)”. Examples of output include “priority=0.98, breakfast=oatmeal+yogurt”, “priority=0.95, dinner=grilled salmon+vegetable soup”, and “priority=0.99, party=roast beef+salad+dessert”. The proposal unit incorporates penalty terms such as “submission timing compatibility”, “event compatibility”, and “user satisfaction” into the AI model's loss function, and updates weights using gradient descent or Adam optimization. Furthermore, the proposal unit can sequentially learn user feedback (e.g., compatibility of proposal timing, satisfaction with event proposals, feasibility, etc.) and personalize the model through online learning. As a technical effect, the proposal unit can automate meal plan proposals at optimal timing in real time according to the user's lifestyle and event schedule, thereby achieving remarkable effects such as improved proposal acceptance rate, user satisfaction, health management accuracy, and system operational efficiency. Specific application fields include personal health management apps, corporate welfare systems, patient meal guidance in medical institutions, event meal management for sports teams, and meal scheduling in nursing care facilities.

The following briefly describes the processing flow of Example of the Embodiment. Specifically, the system is composed of three main modules: the information collection unit, the proposal unit, and the reception unit, each of which cooperates via high-dimensional vector data. The information collection unit acquires user attribute information (e.g., age, gender, health status, meal history, taste preferences, allergy information, lifestyle habits, geographic location, social media activity, emotional state, etc.) through various input interfaces (e.g., text input, voice input, image upload, API integration, etc.), normalizes, categorizes, and quantifies the data, and stores it as multidimensional vectors (e.g., 128 dimensions) in a database. The collection unit uses storage such as time-series databases or NoSQL databases to efficiently manage history data and real-time data. The proposal unit inputs vector data obtained from the collection unit into a Transformer-based large language model or multimodal model, and generates optimal meal plans (e.g., recipe name, cooking steps, nutrient distribution, recommended ingredients, allergen exclusion information, proposal expression parameters, etc.) as structured data, taking into account the user's health goals, emotional state, lifestyle, meal history, and so on. The AI model incorporates penalty terms such as “health goal compatibility”, “emotion compatibility”, “allergen avoidance”, “information overload avoidance”, and “user satisfaction” into the loss function, and updates weights using gradient descent or Adam optimization. The reception unit receives user feedback (e.g., satisfaction, improvement requests, feasibility, selection of input method, etc.) on the meal plan output by the proposal unit through various interfaces, and uses emotion estimation models and history analysis models to automatically adjust the reception method and priority. The overall data flow connects modules via communication methods such as REST API or message queues, and personalizes the model for each user through online learning. As a result, the system achieves remarkable technical effects such as optimization in high-dimensional space, real-time personalization, information overload avoidance, improved user satisfaction, and system operational efficiency, unlike conventional rule-based or human heuristic-based proposal, collection, and reception. Specific application fields include personal health management apps, patient guidance in medical institutions, corporate welfare systems, nutrition management for sports teams, and meal optimization in nursing care facilities.

Step 1: The collection unit collects user information. User information includes, for example, age, gender, health status, and dietary preferences. The collection unit stores the information entered by the user in a database and converts it into a format that is easy for AI to analyze. Step 2: The proposal unit proposes an optimal meal plan based on the information collected by the collection unit. The proposal unit uses AI to generate meal plans based on the user's nutritional needs, taste preferences, and health status. For example, if the user is on a diet, the proposal unit proposes low-calorie and nutritionally balanced meals. If the user has specific allergies, the proposal unit proposes meals that do not contain those allergens. Furthermore, the proposal unit selects recipes and ingredients according to the user's taste preferences. Step 3: The reception unit receives user feedback on the meal plan proposed by the proposal unit. The user can input satisfaction and improvement points regarding the proposed meal plan. The reception unit uses AI to analyze user feedback and provides feedback to the proposal unit. As a result, the proposal unit can improve the meal plan based on user feedback. Specifically, in Step 1, the system normalizes, categorizes, and quantifies user input information (e.g., age=35, gender=female, health status=hypertension, preference=Japanese cuisine, allergy=wheat) and stores it as a multidimensional vector (e.g., 64 dimensions) in a database. The collection unit also accepts multimodal data such as voice input and image upload, and applies preprocessing such as Mel spectrogram conversion for voice and resizing/standardization for images. In Step 2, the proposal unit inputs vector data obtained from the collection unit into a Transformer-based large language model or multimodal model, incorporates penalty terms such as “health goal compatibility”, “allergen avoidance”, and “taste preference compatibility” into the loss function, and generates optimal meal plans (e.g., breakfast=natto rice, lunch=chicken breast salad, dinner=grilled salmon+vegetable soup, calories, nutrient distribution, cooking steps, recommended recipe URL for each meal). Examples of output include “breakfast=oatmeal+yogurt (calories: 250 kcal, protein: 10 g, fat: 5 g, carbohydrates: 40 g)” and “lunch=grilled chicken and vegetables”. In Step 3, the reception unit receives user feedback (e.g., satisfaction=4/5, improvement request=‘want to reduce salt’) via text, voice, or image, automatically adjusts the reception method and priority using emotion estimation models and history analysis models, and returns the feedback content to the proposal unit as structured data. The AI model personalizes the model for each user through online learning, thereby achieving technical effects such as improved proposal accuracy, user satisfaction, and system operational efficiency. Specific application fields include personal health management apps, patient guidance in medical institutions, corporate welfare systems, nutrition management for sports teams, and meal optimization in nursing care facilities.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL:https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned collection unit, proposal unit, and reception unit is implemented, for example, by at least one of the smart device 14 and the data processing apparatus 12. For example, the collection unit collects user information by a control unit 46A of the smart device 14 and converts it into a format that is easy to analyze by a specific processing unit 290 of the data processing apparatus 12. The proposal unit is implemented, for example, by the specific processing unit 290 of the data processing apparatus 12 and generates an optimal meal plan using AI. The reception unit receives user feedback by the control unit 46A of the smart device 14 and analyzes it by the specific processing unit 290 of the data processing apparatus 12. The correspondence between each unit and the device or control unit is not limited to the examples described above and various modifications are possible.

### Second Embodiment

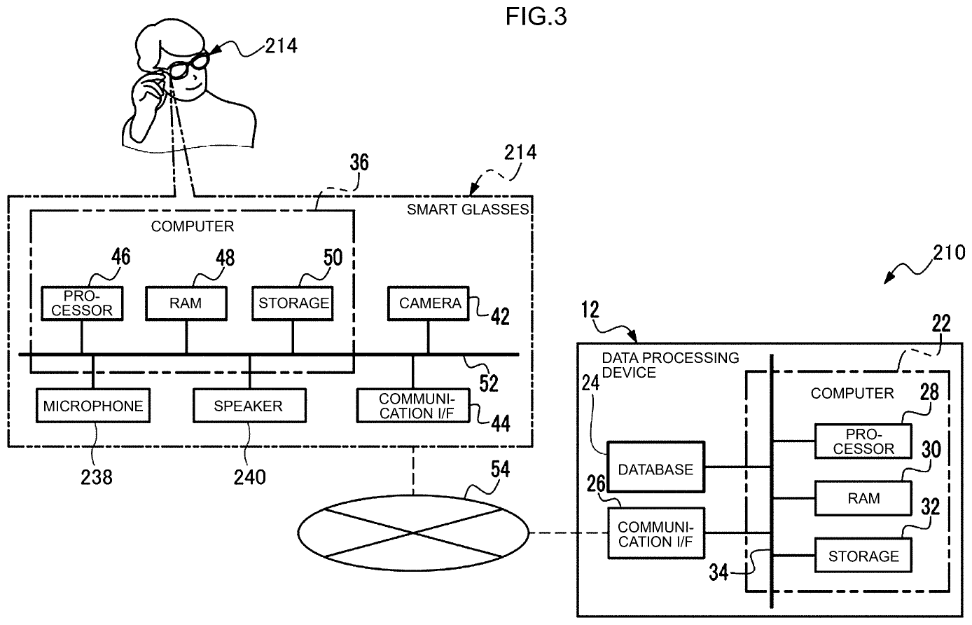

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

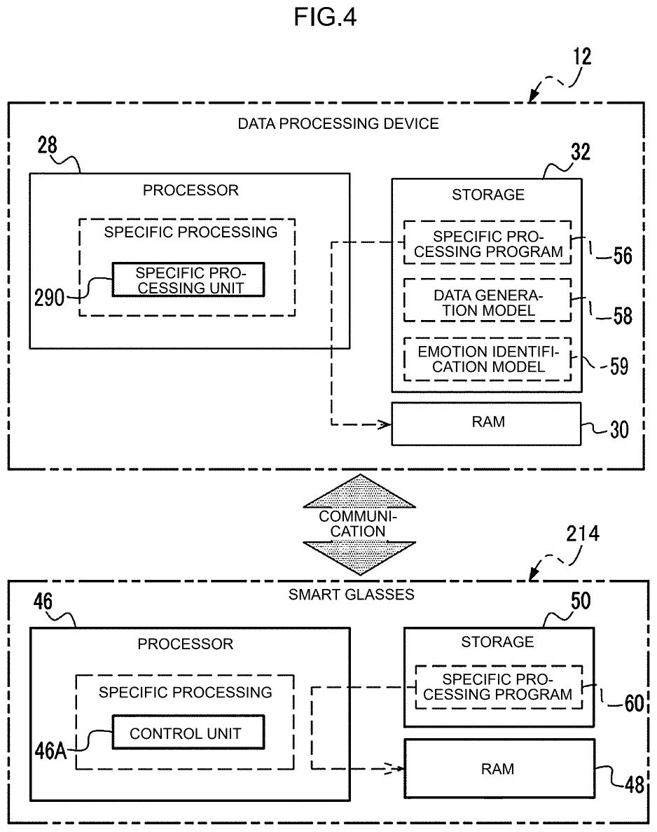

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned collection unit, proposal unit, and reception unit is implemented, for example, by at least one of the smart glasses 214 and the data processing apparatus 12. For example, the collection unit collects user information by a control unit 46A of the smart glasses 214 and converts it into a format that is easy to analyze by a specific processing unit 290 of the data processing apparatus 12. The proposal unit is implemented, for example, by the specific processing unit 290 of the data processing apparatus 12 and generates an optimal meal plan using AI. The reception unit receives user feedback by the control unit 46A of the smart glasses 214 and analyzes it by the specific processing unit 290 of the data processing apparatus 12. The correspondence between each unit and the device or control unit is not limited to the examples described above and various modifications are possible.

### Third Embodiment

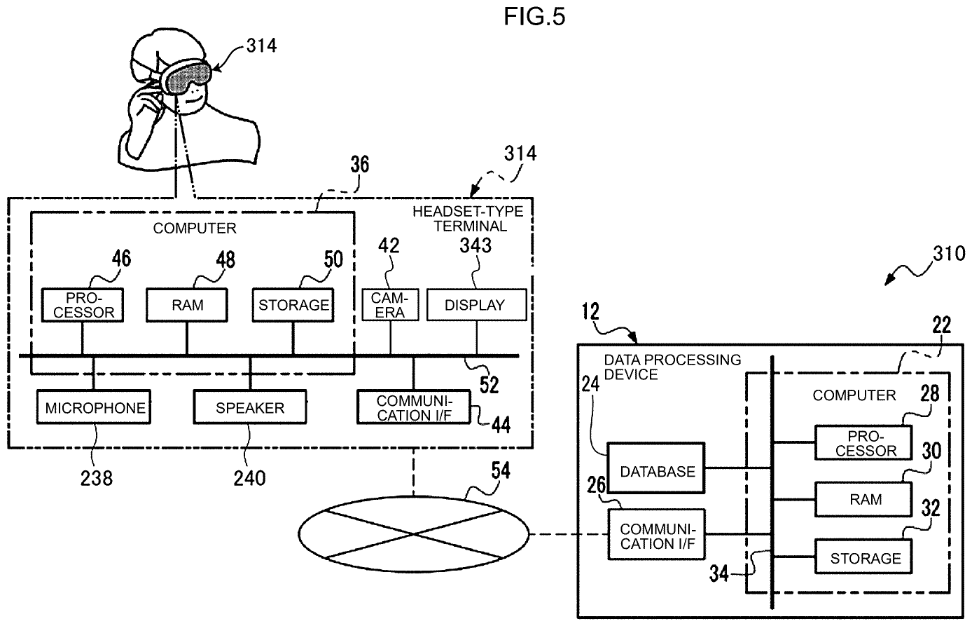

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

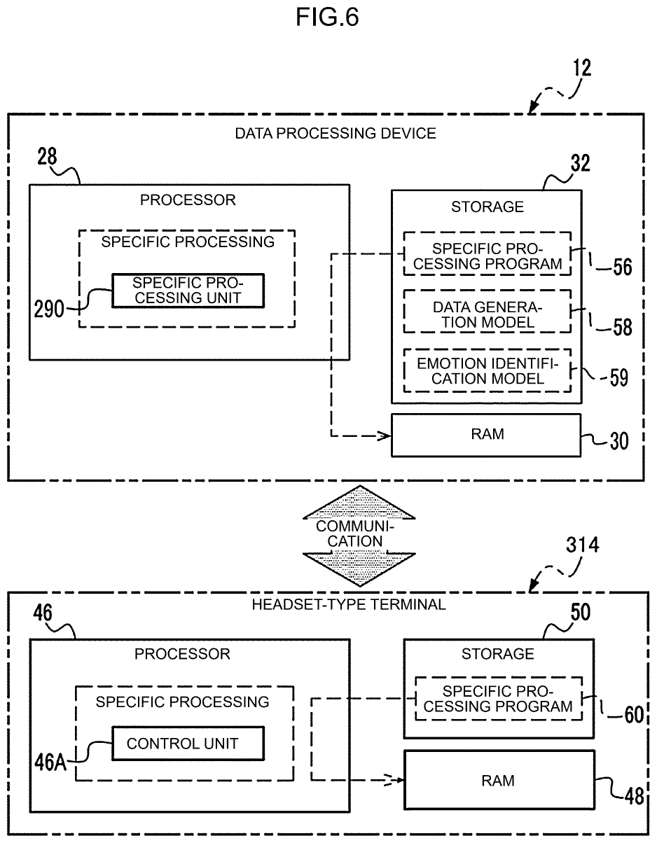

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned collection unit, proposal unit, and reception unit is implemented, for example, by at least one of the headset-type terminal 314 and the data processing apparatus 12. For example, the collection unit collects user information by a control unit 46A of the headset-type terminal 314 and converts it into a format that is easy to analyze by a specific processing unit 290 of the data processing apparatus 12. The proposal unit is implemented, for example, by the specific processing unit 290 of the data processing apparatus 12 and generates an optimal meal plan using AI. The reception unit receives user feedback by the control unit 46A of the headset-type terminal 314 and analyzes it by the specific processing unit 290 of the data processing apparatus 12. The correspondence between each unit and the device or control unit is not limited to the examples described above and various modifications are possible.

### Fourth Embodiment

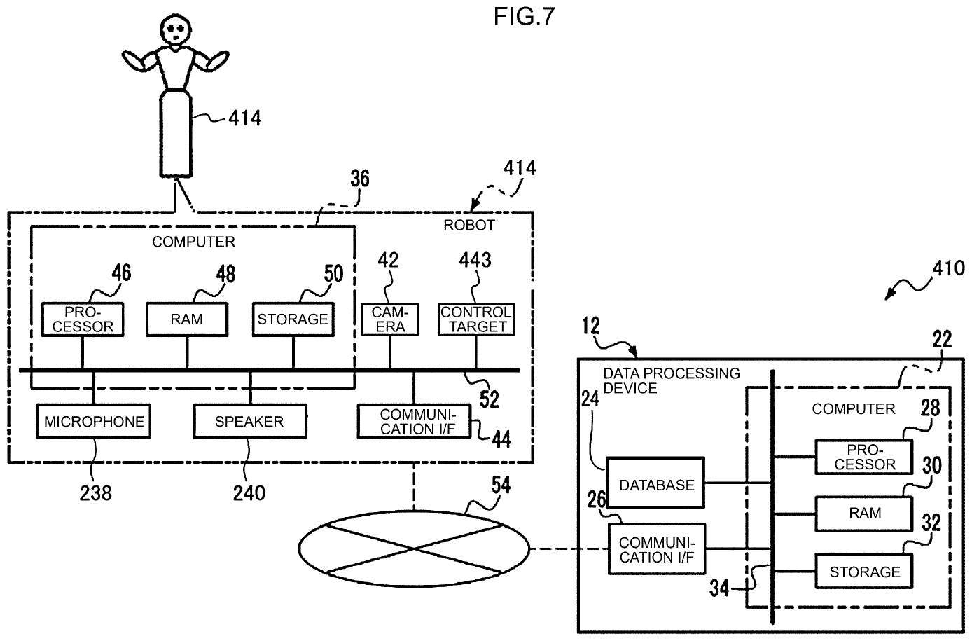

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

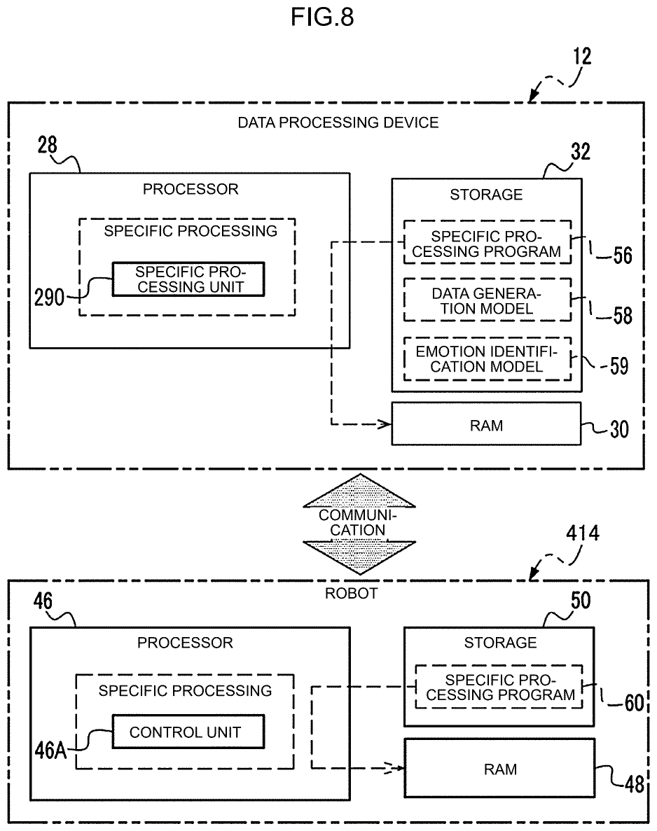

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned collection unit, proposal unit, and reception unit is implemented, for example, by at least one of the robot 414 and the data processing apparatus 12. For example, the collection unit collects user information by a control unit 46A of the robot 414 and converts it into a format that is easy to analyze by a specific processing unit 290 of the data processing apparatus 12. The proposal unit is implemented, for example, by the specific processing unit 290 of the data processing apparatus 12 and generates an optimal meal plan using AI. The reception unit receives user feedback by the control unit 46A of the robot 414 and analyzes it by the specific processing unit 290 of the data processing apparatus 12. The correspondence between each unit and the device or control unit is not limited to the examples described above and various modifications are possible.

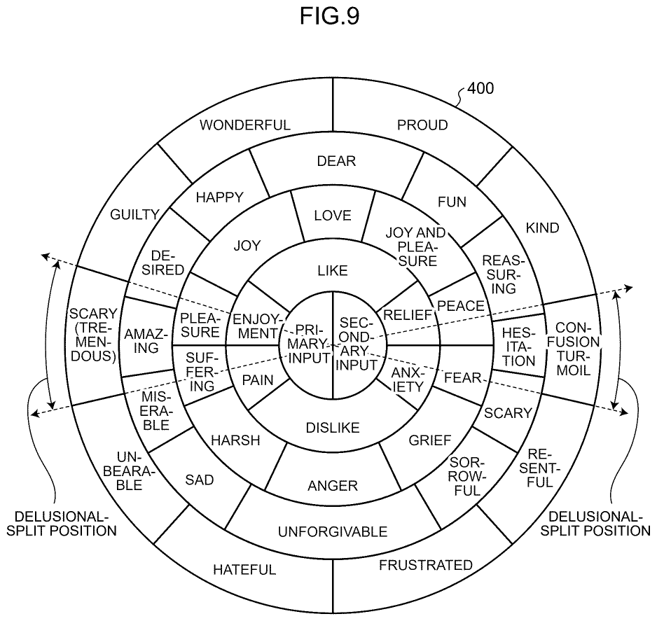

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

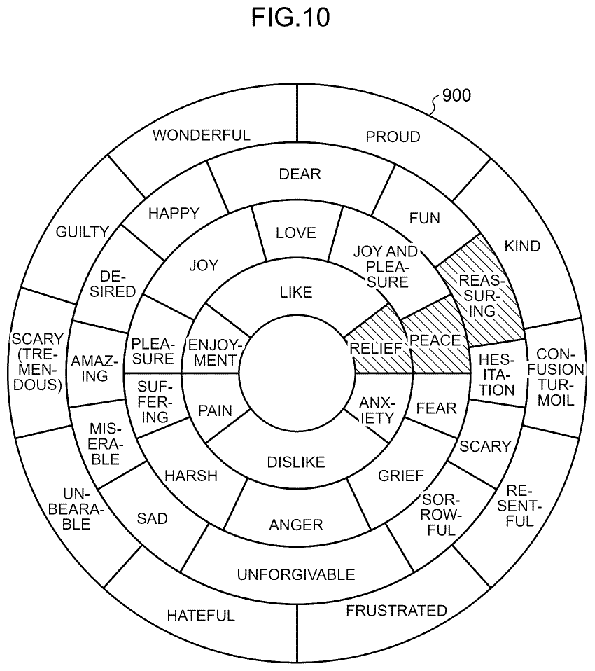

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1) A system comprising: a collection unit configured to collect user information; a proposal unit configured to propose an appropriate meal plan based on the information collected by the collection unit; and a reception unit configured to receive user feedback on the meal plan proposed by the proposal unit.
(Supplementary Note 2) The system according to Supplementary Note 1, wherein the proposal unit is configured to propose an appropriate meal plan based on information regarding the user's age, gender, weight, height, allergy information, dietary preferences, and exercise habits.
(Supplementary Note 3) The system according to Supplementary Note 1, wherein the reception unit is configured to receive user feedback, and the proposal unit is configured to improve the meal plan based on the feedback.
(Supplementary Note 4) The system according to Supplementary Note 1, wherein the proposal unit is configured to select recipes and ingredients tailored to the user's lifestyle.
(Supplementary Note 5) The system according to Supplementary Note 1, wherein the proposal unit is configured to propose low-calorie and nutritionally balanced meals when the user is on a diet.
(Supplementary Note 6) The system according to Supplementary Note 1, wherein the proposal unit is configured to propose meals containing nutrients necessary for training to athletes.
(Supplementary Note 7) The system according to Supplementary Note 1, wherein the collection unit is configured to estimate the user's emotions and adjust the timing of information collection based on the estimated emotions.
(Supplementary Note 8) The system according to Supplementary Note 1, wherein the collection unit is configured to analyze the user's past meal history and select an optimal information collection method.
(Supplementary Note 9) The system according to Supplementary Note 1, wherein the collection unit is configured to perform filtering based on the user's current health status and lifestyle at the time of information collection.
(Supplementary Note 10) The system according to Supplementary Note 1, wherein the collection unit is configured to estimate the user's emotions and determine the priority of information to be collected based on the estimated emotions.
(Supplementary Note 11) The system according to Supplementary Note 1, wherein the collection unit is configured to preferentially collect highly relevant information based on the user's geographic location at the time of information collection.
(Supplementary Note 12) The system according to Supplementary Note 1, wherein the collection unit is configured to analyze the user's social media activity and collect relevant information at the time of information collection.
(Supplementary Note 13) The system according to Supplementary Note 1, wherein the proposal unit is configured to estimate the user's emotions and adjust the manner of presenting proposals based on the estimated emotions.
(Supplementary Note 14) The system according to Supplementary Note 1, wherein the proposal unit is configured to adjust the level of detail of proposals based on the importance of the meal plan at the time of proposal.
(Supplementary Note 15) The system according to Supplementary Note 1, wherein the proposal unit is configured to apply different proposal algorithms according to the category of the meal plan at the time of proposal.
(Supplementary Note 16) The system according to Supplementary Note 1, wherein the proposal unit is configured to estimate the user's emotions and adjust the length of the proposal based on the estimated emotions.
(Supplementary Note 17) The system according to Supplementary Note 1, wherein the proposal unit is configured to determine the priority of proposals based on the submission timing of the meal plan at the time of proposal.
(Supplementary Note 18) The system according to Supplementary Note 1, wherein the proposal unit is configured to adjust the order of proposals based on the relevance of the meal plan at the time of proposal.
(Supplementary Note 19) The system according to Supplementary Note 1, wherein the reception unit is configured to estimate the user's emotions and adjust the method of receiving feedback based on the estimated emotions.
(Supplementary Note 20) The system according to Supplementary Note 1, wherein the reception unit is configured to refer to the user's past feedback history and select an optimal reception method at the time of receiving feedback.
(Supplementary Note 21) The system according to Supplementary Note 1, wherein the reception unit is configured to estimate the user's emotions and determine the priority of feedback based on the estimated emotions.
(Supplementary Note 22) The system according to Supplementary Note 1, wherein the reception unit is configured to consider the user's device information and select an optimal reception method at the time of receiving feedback.

What is claimed is:

1. A system comprising:
circuitry configured to:
receive attribute data of a user from a client terminal;
normalize, categorize, and quantify the attribute data to generate a multidimensional vector;
store the multidimensional vector in a database;
generate output data by inputting the multidimensional vector into a data generation model obtained by machine learning, the data generation model generating the output data as structured data based on constraint parameters incorporated into a loss function of the data generation model;
transmit the output data to the client terminal;
receive response data from the client terminal, the response data indicating an evaluation of the output data by the user;
convert the response data into structured feedback data; and
update weights of the data generation model based on the structured feedback data using online learning to personalize the data generation model for the user.

2. The system according to claim 1, wherein the attribute data comprises at least one of age, gender, weight, height, allergy information, dietary preferences, or exercise habits of the user, and wherein the output data comprises a meal plan.

3. The system according to claim 1, wherein the structured feedback data comprises at least one of a satisfaction score, an implementation status indicator, a taste evaluation, or a request for improvement, and wherein the circuitry is further configured to generate updated output data reflecting the structured feedback data.

4. The system according to claim 1, wherein the attribute data further comprises lifestyle information including at least one of occupation, working hours, family structure, or cooking skill level, and wherein the circuitry is further configured to select, from a plurality of candidate outputs generated by the data generation model, a candidate output matching the lifestyle information.

5. The system according to claim 1, wherein the constraint parameters incorporated into the loss function comprise at least one of a calorie constraint, a nutritional balance constraint, or a taste preference fit parameter.

6. The system according to claim 1, wherein the attribute data further comprises training information including at least one of a sport type, a training frequency, or a training intensity, and wherein the constraint parameters incorporated into the loss function further comprise at least one of a protein target amount or an energy supply parameter.

7. The system according to claim 1, wherein the circuitry is further configured to:
receive multimodal sensor data from the client terminal, the multimodal sensor data comprising at least one of audio data, text data, or image data;
estimate an emotion of the user by inputting the multimodal sensor data into an emotion identification model obtained by machine learning, the emotion identification model generating an emotion label and an emotion intensity score; and
adjust a timing of receiving the attribute data based on the emotion label and the emotion intensity score.

8. The system according to claim 1, wherein the circuitry is further configured to retrieve past attribute data of the user from the database, analyze the past attribute data using at least one of a Transformer-based time-series analysis model or a long short-term memory network, and select an information collection method based on the analysis, the information collection method comprising at least one of a questionnaire format, an image upload, or a voice input.

9. The system according to claim 1, wherein the circuitry is further configured to filter the attribute data based on a current status and a lifestyle pattern of the user prior to generating the multidimensional vector, the filtering comprising selecting attribute categories relevant to the current status from a plurality of attribute categories.

10. The system according to claim 7, wherein the circuitry is further configured to determine a priority of categories of the attribute data to be received based on the emotion label and the emotion intensity score, such that when the emotion intensity score for a stress label exceeds a threshold, the circuitry prioritizes receiving attribute data associated with a relaxation category.

11. The system according to claim 1, wherein the circuitry is further configured to receive geographic location information of the user from the client terminal, and prioritize collection of attribute data associated with a geographic region indicated by the geographic location information.

12. The system according to claim 1, wherein the circuitry is further configured to receive social media activity data of the user from the client terminal, analyze the social media activity data using a multimodal model to identify interest categories, and prioritize collection of attribute data associated with the identified interest categories.

13. The system according to claim 7, wherein the circuitry is further configured to adjust a manner of presenting the output data based on the emotion label and the emotion intensity score, such that when the emotion label indicates stress, the circuitry generates the output data in a simplified format emphasizing concise expressions, and when the emotion label indicates relaxation, the circuitry generates the output data in a detailed format including explanatory information.

14. The system according to claim 1, wherein the circuitry is further configured to compute an importance score for the output data based on the attribute data of the user, and adjust a level of detail of the output data based on the importance score, such that a higher importance score results in the output data including additional parameters comprising at least one of nutrient distribution, procedural steps, or source references.

15. The system according to claim 1, wherein the data generation model comprises a plurality of sub-models, each sub-model associated with a different category label, and wherein the circuitry is further configured to classify the attribute data into a category label and selectively apply a sub-model corresponding to the category label, each sub-model having a different loss function with different constraint parameters.

16. The system according to claim 7, wherein the circuitry is further configured to adjust a length of the output data based on the emotion label and the emotion intensity score by applying an output length adjustment algorithm, such that when the emotion intensity score for a stress label exceeds a threshold, the circuitry generates the output data as a summary of key points, and when the emotion intensity score for a relaxation label exceeds a threshold, the circuitry generates the output data with detailed explanations and background information.

17. The system according to claim 1, wherein the circuitry is further configured to retrieve past response data of the user from the database, analyze the past response data to identify a preferred response format of the user, and transmit, to the client terminal, a feedback interface configured to receive the response data in the preferred response format, the preferred response format comprising at least one of text input, voice input, or image upload.

18. A system comprising:
a communication interface configured to communicate with a client terminal via a packet-switched network;
a processor;
a random access memory;
a memory storing a data generation model obtained by machine learning and an emotion identification model obtained by machine learning;
a database; and
circuitry configured to:
receive, from the client terminal via the communication interface and the packet-switched network, attribute data of a user, the attribute data comprising at least one of age, gender, weight, height, allergy information, dietary preferences, exercise habits, or lifestyle information;
normalize, categorize, and quantify the attribute data to generate a multidimensional vector, and store the multidimensional vector in the database;
receive multimodal sensor data from the client terminal via the communication interface, the multimodal sensor data comprising at least one of audio data captured by a microphone of the client terminal, text data, or image data captured by a camera of the client terminal;
estimate an emotion of the user by inputting the multimodal sensor data into the emotion identification model, the emotion identification model generating an emotion label and an emotion intensity score;
generate output data by inputting the multidimensional vector into the data generation model, the data generation model generating the output data as structured data comprising a meal plan based on constraint parameters incorporated into a loss function of the data generation model, the constraint parameters comprising at least one of a calorie constraint, a nutritional balance constraint, an allergen avoidance constraint, or a taste preference fit parameter;
adjust at least one of a manner of presenting the output data or a length of the output data based on the emotion label and the emotion intensity score;
transmit the output data to the client terminal via the communication interface and the packet-switched network;
receive response data from the client terminal via the communication interface, the response data comprising at least one of a satisfaction score, an implementation status indicator, a taste evaluation, or a request for improvement;
convert the response data into structured feedback data; and
update weights of the data generation model based on the structured feedback data using online learning to personalize the data generation model for the user.

19. The system according to claim 18, wherein the circuitry is further configured to receive device information of the client terminal, the device information comprising at least one of a device type, a screen size, or an input interface type, and select a feedback reception method based on the device information, the feedback reception method comprising at least one of a voice input interface, a touch-optimized interface, or a keyboard input interface.

20. A method performed by circuitry of a system, the method comprising:
receiving attribute data of a user from a client terminal;
normalizing, categorizing, and quantifying the attribute data to generate a multidimensional vector;
storing the multidimensional vector in a database;
generating output data by inputting the multidimensional vector into a data generation model obtained by machine learning, the data generation model generating the output data as structured data based on constraint parameters incorporated into a loss function of the data generation model;
transmitting the output data to the client terminal;
receiving response data from the client terminal, the response data indicating an evaluation of the output data by the user;
converting the response data into structured feedback data; and
updating weights of the data generation model based on the structured feedback data using online learning to personalize the data generation model for the user.
